(19) United States
(12) Patent Application Publication
(10) Pub. No.: US 2021/0198645 A1
(43) Pub. Date: Jul. 1, 2021

(54) NOVEL ANTIMICROBIAL FUSION PROTEINS

(71) Applicant: LYSANDO AG, Triesenberg (LI)

(72) Inventors: Manfred BIEBL, Obertraubling (DE); Martin GRIESSL, Hemau (DE)

(21) Appl. No.: 17/057,746

(22) PCT Filed: May 29, 2019
(86) PCT No.: PCT/EP2019/064097

§ 371 Date: Nov. 23, 2020

(30) Foreign Application Priority Data
May 30, 2018 (EP) 18175064.7

(51) Int. Cl. C12N 9/36; C07K 14/47; A61L 2/16; C07K 14/435

(52) U.S. Cl. CPC C12N 9/2462; C12Y 302/01017; C07K 14/4723; A61K 38/00; C07K 14/43563; C07K 2319/00; A61L 2/16

(57) ABSTRACT

The present invention relates to a polypeptide comprising a Gram negative endolysin and a peptide selected from the group consisting of an antimicrobial peptide, an amphipathic peptide, a cationic peptide, a sushi peptide or a defensin, wherein the endolysin in turn is an endolysin comprising a sequence according to SEQ ID NO:1 or SEQ ID NO:2. The present invention relates also to corresponding nucleic acids, vectors, bacteriophages, host cells, compositions and kits. The present inventions also relates to the use of said polypeptides, nucleic acids, vectors, bacteriophages, host cells, compositions and kits in methods for treatment of the human or animal body by surgery or therapy or in diagnostic methods practiced on the human or animal body. The polypeptides, nucleic acids, vectors, bacteriophages, host cells, compositions and kits according to the invention may also be used as an antimicrobial in, e.g., food or feed, in cosmetics, or as disinfecting agent.

The present invention relates to a polypeptide comprising a Gram negative endolysin and a peptide selected from the group consisting of an antimicrobial peptide, an amphipathic peptide, a cationic peptide, a sushi peptide or a defensin, wherein the endolysin in turn is an endolysin comprising a sequence according to SEQ ID NO:1 or SEQ ID NO:2. The present invention relates also to corresponding nucleic acids, vectors, bacteriophages, host cells, compositions and kits. The present inventions also relates to the use of said polypeptides, nucleic acids, vectors, bacteriophages, host cells, compositions and kits in methods for treatment of the human or animal body by surgery or therapy or in diagnostic methods practiced on the human or animal body. The polypeptides, nucleic acids, vectors, bacteriophages, host cells, compositions and kits according to the invention may also be used as an antimicrobial in, e.g., food or feed, in cosmetics, or as disinfecting agent.

Resistance to conventional antibiotics is becoming an increasing health risk for humankind New antibiotics resistance mechanisms are emerging and rapidly spreading globally. Consequently, the ability to treat common infectious diseases may become more and more difficult in the near future. This danger has been readily understood in the art and new approaches to combat bacterial infectious agents are explored.

Among these new approaches is the creation of fusion proteins combining endolysins with different kinds of peptides. Endolysins are muralytic enzymes (in particular peptidoglycan hydrolases) encoded by bacteriophages (i.e. bacterial viruses). They are synthesized during late gene expression in the lytic cycle of phage multiplication and mediate the release of progeny virions from infected cells through degradation of the bacterial peptidoglycan. In terms of enzymatic activity they are usually either β(1,4)-glycosylases (lysozymes), transglycosylases, amidases or endopeptidases. Antimicrobial application of endolysins was already suggested in 1991. Although the killing capacity of endolysins has been known for a long time, the use of these enzymes as antibacterials was ignored due to the success and dominance of antibiotics. Only after the appearance of multiple antibiotic resistant bacteria this simple concept of combating human pathogens with endolysins received interest. A compelling need to develop totally new classes of antibacterial agents emerged and endolysins used as ‘enzybiotics’—a hybrid term of ‘enzymes’ and ‘antibiotics’—perfectly met this need. In 2001, Fischetti and coworkers demonstrated for the first time the therapeutic potential of bacteriophage Cl endolysin towards group A streptococci. Since then many publications have established endolysins as an attractive and complementary alternative to control bacterial infections, particularly by Gram positive bacteria. Subsequently different endolysins against other Gram positive pathogens such as Streptococcus pneumoniae, Bacillus anthracis, S. agalactiae and Staphylococcus aureus have proven their efficacy as enzybiotics. For a long time then, the most important challenge of endolysin therapy laid in the insensitivity of Gram-negative bacteria towards the exogenous action of endolysins, since the outer membrane shields the access of endolysins from the peptidoglycan.

Gram-negative bacteria possess an outer membrane, with its characteristic asymmetric bilayer as a hallmark. The outer membrane bilayer consists of an inner monolayer containing phospholipids (primarily phosphatidyl ethanolamine) and an outer monolayer that is mainly composed of a single glycolipid, lipopolysaccharide (LPS). There is an immense diversity of LPS structures in the bacterial kingdom and the LPS structure may be modified in response to prevailing environmental conditions. The stability of the LPS layer and interaction between different LPS molecules is mainly achieved by the electrostatic interaction of divalent ions (Mg2+, Ca2+) with the anionic components of the LPS molecule (phosphate groups in the lipid A and the inner core and carboxyl groups of KDO). Furthermore, the dense and ordered packing of the hydrophobic moiety of lipid A, favored by the absence of unsaturated fatty acids, forms a rigid structure with high viscosity. This makes it less permeable for lipophilic molecules and confers additional stability to the outer membrane (OM).

In order to overcome the shielding effect of the outer membrane, endolysins of Gram negative bacteria have been meanwhile successfully fused with, e.g. cationic, amphipathic, hydrophobic or antimicrobial peptides. Such fusion proteins are capable of eliminating Gram negative bacteria when added from without (see for example WO 2010/023207, WO 2010/149792, WO 2011/134998, WO 2012/146738, or WO 2015/121443). However, for achieving improved antibacterial activity, said fusion proteins are frequently combined with small amounts of ethylene diamine tetraacetic acid (EDTA). EDTA is a chelator and known outer membrane permeabilizer (Vaara, M. Microbiol. Rev. 1992 September; 56 (3):395-411, incorporated herein by reference). By removing divalent cations from their binding cites, a disruption of the outer membrane is caused, which typically improves the antibacterial activity of the above mentioned fusion proteins (see for example WO 2010/023207, tables 6 and 8).

While there are various fields of application where the use of EDTA is perfectly acceptable (e.g. in a disinfectant), there are other fields of use where the parallel use of EDTA or other outer membrane permeabilizers is suboptimal or even undesirable (e.g., in the fields of animal feed, food safety, medical devices, and in the pharmaceutical field in general), because EDTA will unspecifically form a complex with any kind of cations, not only those of the bacterial membrane.

Therefore, there is still a need in the art for further improvement in the design of such antibacterial fusion proteins, in particular for applications which do not allow parallel use of EDTA.

The problem to be solved by the present invention was thus to provide new antimicrobial agents of the aforementioned type, which exhibit (in particular under physiological conditions) antibacterial activity and are less dependent on the parallel presence of EDTA or other outer membrane permeabilizing substances.

This problem is solved by the subject-matter as set forth in the appended claims and in the description below.

The inventors of the present invention have surprisingly found that endolysins exhibiting certain sequence motifs (SEQ ID NO:1, or SEQ ID NO:2, respectively) are particularly useful when fused to cationic, amphipathic or antimicrobial peptides. The resulting fusion proteins exhibit a significant antibacterial activity if added from without to Gram negative bacteria such as E. coli, and are, surprisingly, at the same time much less dependent on the parallel presence of EDTA as permeabilizer of the outer membrane of Gram negative bacteria than other antibacterial fusion proteins of this kind. This surprising property renders these polypeptides particularly suited for application in EDTA sensitive fields of use.

The term “polypeptide” as used herein refers in particular to a polymer of amino acid residues linked by peptide bonds in a specific sequence. The amino acid residues of a polypeptide may be modified by e.g. covalent attachments of various groups such as carbohydrates and phosphate. Other substances may be more loosely associated with the polypeptide, such as heme or lipid, giving rise to conjugated polypeptides which are also comprised by the term “polypeptide” as used herein. The term as used herein is intended to encompass also proteins. Thus, the term “polypeptide” also encompasses for example complexes of two or more amino acid polymer chains. The term “polypeptide” does encompass embodiments of polypeptides which exhibit optionally modifications typically used in the art, e.g. biotinylation, acetylation, pegylation, chemical changes of the amino-, SH— or carboxyl-groups (e.g. protecting groups) etc. As will become apparent from the description below, the polypeptide according to the invention is an artificially engineered polypeptide, which does not exist in this form in nature. Such polypeptide may for example exhibit artificial mutations vis-à-vis a naturally occurring polypeptide or may comprise heterologous sequences, or may be a fragment of a naturally occurring polypeptide, which fragment does not occur in this form in nature. Furthermore, the polypeptide according to the present invention is a fusion protein, i.e. represents the linkage of at least two amino acid sequences which do not occur in this combination in nature. The term “polypeptide” as used herein is not limited to a specific length of the amino acid polymer chain. Usually, but not necessarily, a typical polypeptide of the present invention will not exceed about 1000 amino acids in length. The inventive polypeptide may for instance be at most about 750 amino acids long, at most about 500 amino acids long or at most about 300 amino acids long. A possible length range for the inventive polypeptide, without being limited thereto, may thus for example be 16 to 1000 amino acids, 16 to about 50 amino acids, about 200 to about 750 amino acids, or about 225 to about 600 amino acids, or about 250 to about 350 amino acids.

The term “muralytic enzyme”, as used herein, is generally understood in the art. It refers to any polypeptide which is capable of hydrolyzing the peptidoglycan of bacteria, such as Gram negative bacteria. The term is not restricted to a specific enzymatic cleavage mechanism. In terms of cleavage mechanism, the muralytic enzyme may be for example an endopeptidase, chitinase, T4 like muraminidase, lambda like muraminidase, N-acetyl-muramoyl-L-alanine-amidase (amidase), muramoyl-L-alanine-amidase, muramidase, lytic transglycosylase (C), lytic transglycosylase (M), N-acetyl-muramidase (lysozyme), N-acetyl-glucosaminidase or transglycosylases. Furthermore, the term encompasses naturally occurring muralytic enzymes, such as muralytic enzymes (e.g. peptidoglycan hydrolases) of eukaryotic, prokaryotic or viral (in particular bacteriophage) origin. The term encompasses for example vertebrate lysozymes (such as hen egg white lysozyme and human lysozyme), endolysins (e.g. KZ144 endolysin or Lys394 endolysin), Virion-associated peptidoglycan hydrolases (VAPGH), bacteriocins (e.g. lysostaphin) and autolysins. The “muralytic enzyme” may also be a synthetic or artificially modified polypeptide capable of cleaving the peptidoglycan of bacteria. For example, enzymatically active shuffled endolysins in which domains of two or more endolysins have been swapped/exchanged do qualify as “muralytic enzymes” just as truncated endolysins, in which only the enzymatic active domain remains. The activity, in particular of endolysins, can be measured by assays well known in the art by a person skilled in the art as e.g. antibacterial assays which are e.g. described in Briers et al. (J. Biochem. Biophys Methods; 2007; 70: 531-533) or Donovan et al. (J. FEMS Microbiol Lett. 2006 December; 265(1)) (both incorporated herein by reference) and similar publications.

The term “endolysin” as used herein refers to a bacteriophage-derived enzyme which is suitable to catalyze the cleavage (in particular by hydrolysis) of bacterial cell walls. Preferably, endolysins are bacteriophage-derived enzymes which are synthesized by the virus using late gene expression in the lytic cycle of phage multiplication and mediate the release of progeny virions. Endolysins typically exhibit at least one of the following activities: endopeptidase, chitinase, T4 like muraminidase, lambda like muraminidase, N-acetyl-muramoyl-L-alanine-amidase (amidase), muramoyl-L-alanine-amidase, muramidase, lytic transglycosylase (C), lytic transglycosylase (M), N-acetyl-muramidase (lysozyme), N-acetyl-glucosaminidase or transglycosylases as e.g. KZ144 endolysin. In some endolysins, this activity manifests in an individual “enzymatically active domain” (EAD). In addition, the endolysins may contain also regions which are enzymatically inactive, and bind to the cell wall of the host bacteria, the so-called CBDs (cell wall binding domains). The term “endolysin” also encompasses enzymes which comprise modifications and/or alterations vis-a-vis naturally occurring endolysins. Such alterations and/or modifications may comprise mutations such as deletions, insertions and additions, substitutions or combinations thereof and/or chemical changes of the amino acid residues. Particularly preferred chemical changes are biotinylation, acetylation, pegylation, chemical changes of the amino-, SH— or carboxyl-groups. Said endolysins exhibit on a general level the lytic activity of the respective wild-type endolysin. However, said activity can be the same, higher or lower as the activity of the respective wild-type endolysin. Said activity can be for example at least about 50, 60, 70, 80, 90, 100, 110, 120, 130, 140, 150, 160, 170, 180, 190 or at least about 200% of the activity of the respective wild-type endolysin or even more. The activity can be measured by assays well known in the art by a person skilled in the art as e.g. the plate lysis assay or the liquid lysis assay which are e.g. described in Briers et al. (J. Biochem. Biophys Methods; 2007; 70: 531-533) or Donovan et al. (J. FEMS Microbiol Lett. 2006 December; 265(1) (both incorporated herein by reference) and similar publications.

The term “Gram negative endolysin” refers to endolysins deriving from bacteriophages targeting Gram negative bacteria. These endolysins are capable of degrading the peptidoglycan of the respective (host) bacteria.

A “modular” endolysin, as used herein, is an endolysin which exhibits at least two distinct functional domains, namely at least one “enzymatically active domain” (EAD) and at least one “cell-wall-binding domain” (CBD). While the former provides the actual enzymatic activity, the latter may provide for target binding. Due to their domain character, these two activities can be separated from each other. Endolysins lacking a distinct CBD do not fall under the term “modular endolysin”.

The term “bacteriophage tail/baseplate protein” is generally understood be a person skilled in the art. Tail proteins and baseplate proteins are proteins of bacteriophages. Binding structures located in the tail fiber and/or baseplate of bacteriophages play an important role in mediating injection of the phage genome into the host cell. Tail fiber proteins are positioned at the tip of the tail and are responsible for binding to the cell surface by recognizing a potential host bacterium and attaching to its outer surface. Baseplate proteins control the transfer of the genetic material and can have also cell binding properties. Especially for Myoviruses of Gram negative bacteria (e.g. T4 or P2 phages) different motifs are described which show homology to peptidoglycan binding domains like LysM. Another example is the gp5 of the ICP1 Vibrio phage and related proteins encoded in the genome of phages infecting different species like e.g. Methylobacter sp. These consist of a peptidoglycan binding domain and an enzymatic active domain, able to degrade the murein layer of the host bacteria.

The term, “antimicrobial peptide” (AMP) as used herein refers preferably to any peptide that has microbicidal and/or microbistatic activity on, for example, bacteria, viruses, fungi, yeasts, mycoplasma and protozoa. In some embodiments, the peptide will be a naturally occurring peptide. In other embodiments, the peptide will be an artificial peptide not occurring in nature. For example, the antimicrobial peptide may be a mutated version of naturally occurring peptide. The term “antimicrobial peptide” as used herein refers in particular to any peptide having anti-bacterial, anti-fungal, anti-mycotic, anti-parasitic, anti-protozoal, anti-viral, anti-infectious, anti-infective and/or germicidal, algicidal, amoebicidal, microbicidal, bactericidal, fungicidal, parasiticidal, protozoacidal, protozoicidal properties. Preferred are anti-bacterial peptides. The antimicrobial peptide may be a member of the RNase A super family, a defensin, cathelicidin, granulysin, histatin, psoriasin, dermicidine or hepcidin. The antimicrobial peptide may be naturally occurring in insects, fish, plants, arachnids, vertebrates or mammals. Preferably the antimicrobial peptide may be naturally occurring in radish, silk moth, wolf spider, frog, preferably in Xenopus laevis, Rana frogs, more preferably in Rana catesbeiana, toad, preferably Asian toad Bufo bufo gargarizans, fly, preferably in Drosophila, more preferably in Drosophila melanogaster, in Aedes aegypti, in honey bee, bumblebee, preferably in Bombus pascuorum, flesh fly, preferably in Sarcophaga peregrine, scorpion, horseshoe crab, catfish, preferably in Parasilurus asotus, cow, pig, sheep, porcine, bovine, monkey and human. As used herein, an “antimicrobial peptide” (AMP) may in particular be a peptide which is not a cationic peptide, polycationic peptide, amphipathic peptide, sushi peptide or defensins, but nevertheless exhibits antimicrobial activity. Examples of antimicrobial peptides may be found in “The Antimicrobial Peptide Database” of the University of Nebraska Medical Center (Omaha, Nebr., USA; http://aps.unmc.edu/AP/main.php).

The term “amphipathic peptide” as used herein refers to synthetic peptides having both hydrophilic and hydrophobic functional groups. Preferably, the term “amphipathic peptide” as used herein refers to a peptide having a defined arrangement of hydrophilic and hydrophobic groups.

As used herein, the term “cationic peptide” refers to a peptide having positively charged amino acid residues. Preferably a cationic peptide has a pKa-value of 9.0 or greater. Typically, at least four of the amino acid residues of the cationic peptide can be positively charged, for example, lysine or arginine. “Positively charged” refers to the side chains of the amino acid residues which have a net positive charge at about physiological conditions. The term “cationic peptide” as used herein refers also to polycationic peptides, but also includes cationic peptides which comprise for example less than 20%, preferably less than 10% positively charged amino acid residues.

The term “polycationic peptide” as used herein refers preferably to a peptide composed of mostly positively charged amino acid residues, in particular lysine and/or arginine residues. A peptide is composed of mostly positively charged amino acid residues if at least about 60, 70, 75, 80, 85, 90, 95 or about 100% of the amino acid residues are positively charged amino acid residues, in particular lysine and/or arginine residues. The amino acid residues being not positively charged amino acid residues can be neutrally charged amino acid residues and/or negatively charged amino acid residues and/or hydrophobic amino acid residues. Preferably the amino acid residues being not positively charged amino acid residues are neutrally charged amino acid residues, in particular serine and/or glycine.

The term “sushi peptide” as used herein refers to complement control proteins (CCP) having short consensus repeats. The sushi module of sushi peptides functions as a protein-protein interaction domain in many different proteins. Peptides containing a Sushi domain have been shown to have antimicrobial activities. Preferably, sushi peptides are naturally occurring peptides.

The term “defensin” as used herein refers to a peptide present within animals, preferably mammals, more preferably humans, wherein the defensin plays a role in the innate host defense system as the destruction of foreign substances such as infectious bacteria and/or infectious viruses and/or fungi. A defensin is a non-antibody microbicidal and/or tumoricidal protein, peptide or polypeptide. Examples for “defensins” are “mammalian defensins,” alpha-defensins, beta-defensins, indolicidin and magainins The term “defensins” as used herein refers both to an isolated form from animal cells or to a synthetically produced form, and refers also to variants which substantially retain the cytotoxic activities of their parent proteins, but whose sequences have been altered by insertion or deletion of one or more amino acid residues.

As used herein, the term “tag” refers to an amino acid sequence, which is typically in the art fused to or included in another amino acid sequence for a) improving expression of the overall amino acid sequence or polypeptide, b) facilitating purification of the overall amino acid sequence or polypeptide, c) facilitating immobilisation of the overall amino acid sequence or polypeptide, and/or d) facilitating detection of the overall amino acid sequence or polypeptide. Examples for tags are His tags, such as His5-tags, His6-tags, His7-tags, His8-tags, His9-tags, His10-tags, His11-tags, His12-tags, His16-tags and His20-tags, Strep-tags, Avi-tags, Myc-tags, GST-tags, JS-tags, cystein-tags, FLAG-tags, HA-tags, thioredoxin or maltose binding proteins (MBP), CAT, GFP, YFP, etc. The person skilled in the art will know a vast number of tags suitable for different technical applications. The tag may for example make such tagged polypeptide suitable for e.g. antibody binding in different ELISA assay formats or other technical applications.

As used herein, the term “% sequence identity”, has to be understood as follows: Two sequences to be compared are aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length. In the above context, an amino acid sequence having a “sequence identity” of at least, for example, 95% to a query amino acid sequence, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted. Methods for comparing the identity and homology of two or more sequences are well known in the art. The percentage to which two sequences are identical can for example be determined by using a mathematical algorithm. A preferred, but not limiting, example of a mathematical algorithm which can be used is the algorithm of Karlin et al. (1993), PNAS USA, 90:5873-5877. Such an algorithm is integrated in the BLAST family of programs, e.g. BLAST or NBLAST program (see also Altschul et al., 1990, J. Mol. Biol. 215, 403-410 or Altschul et al. (1997), Nucleic Acids Res, 25:3389-3402), accessible through the home page of the NCBI at world wide web site ncbi.nlm.nih.gov) and FASTA (Pearson (1990), Methods Enzymol. 83, 63-98; Pearson and Lipman (1988), Proc. Natl. Acad. Sci. U. S. A 85, 2444-2448.). Sequences which are identical to other sequences to a certain extent can be identified by these programmes. Furthermore, programs available in the Wisconsin Sequence Analysis Package, version 9.1 (Devereux et al, 1984, Nucleic Acids Res., 387-395), for example the programs BESTFIT and GAP, may be used to determine the % identity between two polypeptide sequences. BESTFIT uses the “local homology” algorithm of (Smith and Waterman (1981), J. Mol. Biol. 147, 195-197.) and finds the best single region of similarity between two sequences. If herein reference is made to an amino acid sequence sharing a particular extent of sequence identity to a reference sequence, then said difference in sequence is preferably due to conservative amino acid substitutions. Preferably, such sequence retains the activity of the reference sequence, e.g. albeit maybe at a slower rate. In addition, if reference is made herein to a sequence sharing “at least” at certain percentage of sequence identity, then 100% sequence identity are preferably not encompassed.

The term “comprising”, as used herein, shall not be construed as being limited to the meaning “consisting of” (i.e. excluding the presence of additional other matter). Rather, “comprising” implies that optionally additional matter may be present. The term “comprising” encompasses as particularly envisioned embodiments falling within its scope “consisting of” (i.e. excluding the presence of additional other matter) and “comprising but not consisting of” (i.e. requiring the presence of additional other matter), with the former being more preferred.

The use of the word “a” or “an”, when used herein, may mean “one,” but it is also consistent with the meaning of “one or more,” “at least one,” and “one or more than one.”

The present invention relates in a first aspect to a polypeptide comprising a Gram negative endolysin and a peptide selected from the group consisting of an antimicrobial peptide, an amphipathic peptide, a cationic peptide, a hydrophobic peptide, a sushi peptide or a defensin, wherein the endolysin in turn is an endolysin comprising a sequence according to SEQ ID NO:1, and with preferably the additional provisos that

a) the polypeptide does neither comprise the sequence according to SEQ ID NO:3 nor according to SEQ ID NO:4 nor according to SEQ ID NO:5,

b) the endolysin is neither Aeh1p339 of Aeromonas phage Aeh1 (e.g. NP_944217.1) nor EpJS98_gp116 of Enterobacteria phage J598 (e.g. YP_001595245.1),

c) the peptide is selected from the group consisting of an antimicrobial peptide, an amphipathic peptide, a sushi peptide or a defensin, if the polypeptide comprises the sequence of SEQ ID NO:6,

d) the polypeptide does not comprise a cell wall binding domain of i) a modular Gram-negative endolysin or ii) a bacteriophage tail/baseplate protein, if the endolysin has a sequence selected from the group consisting of:

| Host | Phage name | Protein ID |
|---|---|---|
| Aeromonas | Aeromonas phage PX29 | ADQ53036.1 |
| Aeromonas | Aeromonas phage phiAS4 | YP_003969055.1 |
| Aeromonas | Aeromonas phage 44RR2.8t | NP_932578.1 |
| Aeromonas | Aeromonas phage 25 | YP_656449.1 |
| Aeromonas | Aeromonas phage 31 | YP_238949.1 |
| Aeromonas | Aeromonas phage 65 | YP_004300997.1 |
| Aeromonas | Aeromonas phage phiAS5 | YP_003969406.1 |
| Escherichia | Escherichia phage wV7 | AEM00790.1 |
| Escherichia | Enterobacteria phage vB_EcoM-VR7 | YP_004063811.1 |
| Escherichia | Enterobacteria phage Bp7 | AEN93735.1 |
| Escherichia | Enterobacteria phage AR1 | BAI83135.1 |
| Escherichia | Enterobacteria phage JS10 | YP_002922463.1 |
| Escherichia | Enterobacteria phage IME08 | YP_003734260.1 |
| Escherichia | Enterobacteria phage CC31 | YP_004009990.1 |
| Escherichia | Enterobacteria phage RB69 | NP_861818.1 |
| Escherichia | Enterobacteria phage RB14 | YP_002854463.1 |
| Escherichia | Enterobacteria phage RB32 | ABI94948.1 |
| Escherichia | Enterobacteria phage RB51 | YP_002854084.1 |
| Shigella | Shigella phage Shfl2 | YP_004415022.1 |

and corresponding sequences merely lacking in addition the N-terminal methionine,

e) the polypeptide does not comprise a cell wall binding domain of i) a modular Gram-negative endolysin or ii) a bacteriophage tail/baseplate protein, if the endolysin has a sequence according to amino acids 2-164 of:

| Host | Phage name | Protein ID |
|---|---|---|
| Escherichia | Enterobacteria phage T4 | NP_049736.1 |

f) the polypeptide does not comprise a cell wall binding domain of i) a modular Gram-negative endolysin or ii) a bacteriophage tail/baseplate protein, if the endolysin has a sequence according to amino acids 2-165 of:

| Host | Phage name | Protein ID |
|---|---|---|
| Aeromonas | Aeromonas phage Aeh1 | NP_944217.1 |

g) the polypeptide does not comprise a cell wall binding domain of i) a modular Gram-negative endolysin or ii) a bacteriophage tail/baseplate protein, if the endolysin has a sequence according to amino acids 2-161 of:

| Host | Phage name | Protein ID |
|---|---|---|
| Escherichia | Enterobacteria phage JS98 | YP_001595245.1 |

The present invention relates in a second aspect to a polypeptide comprising a Gram negative endolysin and a peptide selected from the group consisting of an antimicrobial peptide, an amphipathic peptide, a cationic peptide, a hydrophobic peptide, a sushi peptide or a defensin, wherein the endolysin in turn is an endolysin comprising a sequence according to SEQ ID NO:2 (in particular comprising a sequence according to SEQ ID NO:7), and with preferably the additional provisos that

a) the polypeptide does neither comprise the sequence according to SEQ ID NO:3 nor according to SEQ ID NO:4 nor according to SEQ ID NO:5,

b) the endolysin is not EpJS98_gp116 of Enterobacteria phage J598 (e.g. YP_001595245.1),

c) the peptide is selected from the group consisting of an antimicrobial peptide, an amphipathic peptide, a sushi peptide or a defensin, if the polypeptide comprises the sequence of SEQ ID NO:6,

d) the polypeptide does not comprise a cell wall binding domain of i) a modular Gram-negative endolysin or ii) a bacteriophage tail/baseplate protein, if the endolysin has a sequence selected from the group consisting of:

| Host | Phage name | Protein ID |
|---|---|---|
| Aeromonas | Aeromonas phage 65 | YP_004300997.1 |
| Escherichia | Escherichia phage wV7 | AEM00790.1 |
| Escherichia | Enterobacteria phage vB_EcoM-VR7 | YP_004063811.1 |
| Escherichia | Enterobacteria phage Bp7 | AEN93735.1 |
| Escherichia | Enterobacteria phage AR1 | BAI83135.1 |
| Escherichia | Enterobacteria phage JS10 | YP_002922463.1 |
| Escherichia | Enterobacteria phage IME08 | YP_003734260.1 |
| Escherichia | Enterobacteria phage CC31 | YP_004009990.1 |
| Escherichia | Enterobacteria phage RB69 | NP_861818.1 |
| Escherichia | Enterobacteria phage RB14 | YP_002854463.1 |
| Escherichia | Enterobacteria phage RB32 | ABI94948.1 |
| Escherichia | Enterobacteria phage RB51 | YP_002854084.1 |
| Shigella | Shigella phage Shfl2 | YP_004415022.1 |

and corresponding sequences merely lacking in addition the N-terminal methionine,

e) the polypeptide does not comprise a cell wall binding domain of i) a modular Gram-negative endolysin or ii) a bacteriophage tail/baseplate protein, if the endolysin has a sequence according to amino acids 2-164 of:

| Host | Phage name | Protein ID |
|---|---|---|
| Escherichia | Enterobacteria phage T4 | NP_049736.1 |

f) the polypeptide does not comprise a cell wall binding domain of i) a modular Gram-negative endolysin or ii) a bacteriophage tail/baseplate protein, if the endolysin has a sequence according to amino acids 2-161 of:

| Host | Phage name | Protein ID |
|---|---|---|
| Escherichia | Enterobacteria phage JS98 | YP_001595245.1 |

In a third aspect the present invention relates to a polypeptide comprising a Gram negative endolysin and a peptide selected from the group consisting of an antimicrobial peptide, an amphipathic peptide, a cationic peptide, a hydrophobic peptide, a sushi peptide or a defensin, wherein the endolysin in turn is an endolysin comprising a sequence according to SEQ ID NO:1, and wherein the endolysin does not comprise any cysteine residue in its sequence, and preferably with the additional provisos that:

a) the endolysin is not Aehlp339 of Aeromonas phage Aeh1,

b) the polypeptide does not comprise a cell wall binding domain of i) a modular Gram-negative endolysin or ii) a bacteriophage tail/baseplate protein, if the endolysin has a sequence selected from the group consisting of:

| Host | Phage name | Protein ID |
|---|---|---|
| Aeromonas | Aeromonas phage PX29 | ADQ53036.1 |
| Aeromonas | Aeromonas phage 65 | YP_004300997.1 |
| Aeromonas | Aeromonas phage phiAS5 | YP_003969406.1 |

and corresponding sequences merely lacking in addition the N-terminal methionine,

c) the polypeptide does not comprise a cell wall binding domain of i) a modular Gram-negative endolysin or ii) a bacteriophage tail/baseplate protein, if the endolysin has a sequence according to amino acids 2-165 of:

| Host | Phage name | Protein ID |
|---|---|---|
| Aeromonas | Aeromonas phage Aeh1 | NP_944217.1 |

The absence of any cysteine residue in the endolysin sequence may be because already the wildtype form of the endolysin does not comprise such cysteine residue or because any cysteine residues occurring in the wildtype sequence have been technically modified/mutated (e.g. C→S, or C→A or C→G, preferably C→S), for instance to increase stability and to reduce aggregation.

In a fourth aspect the present invention relates to a polypeptide comprising a Gram negative endolysin and a peptide selected from the group consisting of an antimicrobial peptide, an amphipathic peptide, a cationic peptide, a hydrophobic peptide, a sushi peptide or a defensin, wherein the endolysin in turn is an endolysin comprising a sequence according to SEQ ID NO:2 (in particular comprising a sequence according to SEQ ID NO:7), and wherein the endolysin does not comprise any cysteine residue in its sequence, and preferably with the additional provisos that:

the polypeptide does not comprise a cell wall binding domain of i) a modular Gram-negative endolysin or ii) a bacteriophage tail/baseplate protein, if the endolysin has a sequence selected from the group consisting of:

| Host | Phage name | Protein ID |
|---|---|---|
| Aeromonas | Aeromonas phage 65 | YP_004300997.1 |

and a corresponding sequence merely lacking in addition the N-terminal methionine,

The absence of any cysteine residue in the endolysin sequence may be again because already the wildtype form of the endolysin does not comprise such cysteine residue or because any cysteine residues occurring in the wildtype sequence have been technically modified/mutated (e.g. C→S, or C→A or C→G, preferably C→S), for instance to increase stability and to reduce aggregation.

In a fifth aspect the present invention relates to a polypeptide comprising a Gram negative endolysin and a peptide selected from the group consisting of an antimicrobial peptide, an amphipathic peptide, a cationic peptide, a sushi peptide or a defensin, wherein the endolysin in turn is an endolysin comprising a sequence according to SEQ ID NO:1, and wherein the peptide is positioned within the polypeptide N-terminally of the endolysin (for instance in embodiments wherein the endolysin constitutes the most C-terminal component of the polypeptide), and preferably with the additional provisos that the polypeptide does neither comprise the sequence according to SEQ ID NO:4 nor according to SEQ ID NO:5.

In a sixth aspect the present invention relates to a polypeptide comprising a Gram negative endolysin and a peptide selected from the group consisting of an antimicrobial peptide, an amphipathic peptide, a cationic peptide, a sushi peptide or a defensin, wherein the endolysin in turn is an endolysin comprising a sequence according to SEQ ID NO:2 (in particular comprising a sequence according to SEQ ID NO:7), and wherein the peptide is positioned within the polypeptide N-terminally of the endolysin (for instance in embodiments wherein the endolysin constitutes the most C-terminal component of the polypeptide), and preferably with the additional provisos that the polypeptide does neither comprise the sequence according to SEQ ID NO:4 nor according to SEQ ID NO:5.

In the following, the polypeptide of the invention (be it now of the first, second, third, fourth, fifth or sixth aspect of the invention) will be discussed in more detail. It is understood that anything set forth below applies equally to all six of the aspects above, unless explicitly stated otherwise.

The endolysin component of the inventive polypeptide comprises a sequence according to SEQ ID NO:1 or SEQ ID NO:2 (in particular comprising a sequence according to SEQ ID NO:7). SEQ ID NO:1 is 13 amino acids long and has the sequence X₁NRAX₂RVX₃X₄X₅X₆X₇X₈, wherein X₁ may be P or T, X₂ may be K, M, N or Q, X₃ may be A, I or T, X₄ may be A, D, E, K, Q, S, or T, X₅ may be T or V, X₆ may be F, I, L or V, X₇ may be E, K, L or R, X₈ may be L or T. SEQ ID NO:7 is also 13 amino acids long and has the sequence X₁NRAKRVX₂X₃X₄X₅X₆X₇, wherein X₁ may be P or T, X₂ may be A, I or T, X₃ may be A, D, E, or S, X₄ may be T or V, X₅ may be F, I, or L, X₆ may be E, K or R, X₇ may be L or T. The endolysin can be for instance either a naturally occurring endolysin exhibiting such sequence element in its sequence naturally, or is a modified (and thus no longer naturally occurring) endolysin exhibiting such sequence (e.g. an endolysin deriving from a naturally occurring endolysin, which has however been modified by mutation, truncation or the like, for instance to increase activity, stability, expression, cloning reasons etc.). In either case the sequence according to SEQ ID NO:1 (or SEQ ID NO:2 or SEQ ID NO:7, respectively) is an integral part of the endolysin. In contrast thereto, the phrase “endolysin comprising a sequence according to SEQ ID NO:1” (or SEQ ID NO:2 or SEQ ID NO:7, respectively) is preferably not intended to cover a situation in which a sequence element comprising SEQ ID NO:1 (or SEQ ID NO:2 or SEQ ID NO:7, respectively) has been artificially combined with an otherwise entirely unrelated endolysin or EAD. In other words, the sequence according to SEQ ID NO:1 (or SEQ ID NO:2 or SEQ ID NO:7, respectively) is not heterologous to the actual endolysin. SEQ ID NO:1 (like SEQ ID NO:7, respectively) is a consensus sequence indicating possible positions of variation. Typically, the sequence according to SEQ ID NO:1 (or SEQ ID NO:7) will be found in the C-terminal part of the endolysin sequence, e.g., within the range of the last 40, the last 30, or even the last 25 amino acids at the C-terminus of the endolysin sequence. A preferred form of the consensus sequence according to SEQ ID NO:1 is SEQ ID NO:8. An even more preferred form is SEQ ID NO:9. Likewise particularly preferred forms of SEQ ID NO:1 are SEQ ID NO:10 and SEQ ID NO:11. SEQ ID NO:1 may also take the form of SEQ ID NO:12, SEQ ID NO:13 or SEQ ID NO:14. However, in preferred embodiments the polypeptide does not comprise SEQ ID NO:12, SEQ ID NO:13 and/or SEQ ID NO:14. Particularly preferred examples of SEQ ID NO:1 and SEQ ID NO:7, respectively, are SEQ ID NO:15, SEQ ID NO:16, SEQ ID NO:17, SEQ ID NO:18, SEQ ID NO:19 and SEQ ID NO:20.The endolysin derives from a phage infecting Gram negative bacteria, i.e. is a Gram negative endolysin. Examples of endolysins comprising a sequence according to SEQ ID NO:1 (or SEQ ID NO:2 or SEQ ID NO:7, respectively) are for instance the endolysins of Citrobacter koseri phage CkP1 (SEQ ID NO:21) Enterobacteria phage CC31 (SEQ ID NO:22), Serratia phage CHI14 (SEQ ID NO:23), Aeromonas phage Ah1 (SEQ ID NO:24), Serratia phage PS2 (SEQ ID NO:25), and Aeromonas phage AS-szw (SEQ ID NO:26); or sequences sharing at least 80% sequence identity, preferably at least 85% sequence identity, more preferably at least 90% sequence identity, more preferably at least 95% sequence identity, more preferably at least 96% sequence identity, more preferably at least 97% sequence identity, more preferably at least 98% sequence identity, more preferably at least 99% sequence identity with SEQ ID NO:21, SEQ ID NO:22, SEQ ID NO:23, SEQ ID NO:24, SEQ ID NO:25, and SEQ ID NO:26, respectively. Particularly preferred are sequences sharing at least 80% sequence identity, preferably at least 85% sequence identity, more preferably at least 90% sequence identity, more preferably at least 95% sequence identity, more preferably at least 96% sequence identity, more preferably at least 97% sequence identity, more preferably at least 98% sequence identity, more preferably at least 99% sequence identity with SEQ ID NO:21 and/or SEQ ID NO:22. Another suitable endolysin for use in the inventive polypeptide is a derivative of T4 endolysin, e.g. a sequence according to SEQ ID NO:27 or a sequence sharing at least 80% sequence identity, preferably at least 85% sequence identity, more preferably at least 90% sequence identity, more preferably at least 95% sequence identity, more preferably at least 96% sequence identity, more preferably at least 97% sequence identity, more preferably at least 98% sequence identity, more preferably at least 99% sequence identity with SEQ ID NO:27 (subject to the proviso regarding SEQ ID NO:3 forth above). For example, modified versions of these endolysins, with a cysteine replaced by, e.g. a serine (e.g. C54S or C122S, respectively), and/or lacking the N-terminal methionine, are preferred forms of endolysin to be used as component of the inventive polypeptide (see for instance SEQ ID NO:6, subject to the disclaimer above, SEQ ID NO:28, SEQ ID NO:29, SEQ ID NO:30, SEQ ID NO:31, SEQ ID NO:32, SEQ ID NO:33, SEQ ID NO:34, SEQ ID NO:35, and SEQ ID NO:36, which are particularly preferred embodiments). Particularly preferred endolysin sequences are thus also SEQ ID NO:6 (subject to the proviso above), SEQ ID NO:28, SEQ ID NO:29, SEQ ID NO:30, SEQ ID NO:31, SEQ ID NO:32, SEQ ID NO:33, SEQ ID NO:34, SEQ ID NO:35, and SEQ ID NO:36, or a sequence having at least 80% sequence identity, preferably at least 85% sequence identity, more preferably at least 90% sequence identity, more preferably at least 95% sequence identity, more preferably at least 96% sequence identity, more preferably at least 97% sequence identity, more preferably at least 98% sequence identity, more preferably at least 99% sequence identity with SEQ ID NO:6 (subject to the proviso above), SEQ ID NO:28, SEQ ID NO:29, SEQ ID NO:30, SEQ ID NO:31, SEQ ID NO:32, SEQ ID NO:33, SEQ ID NO:34, SEQ ID NO:35, and/or SEQ ID NO:36, in particular to SEQ ID NO:28 and/or SEQ ID NO:30.

Other examples for suitable endolysin components (in particular for the first, third and fifth aspect of the invention and always subject to the above mentioned provisos, and not limited thereto) are listed in table 1 below.

TABLE 1
Examples for suitable endolysin components
in the inventive polypeptide:

| Host | Phage name | Protein ID |
|---|---|---|
| Aeromonas | Aeromonas phage PX29 | ADQ53036.1 |
| Aeromonas | Aeromonas phage phiAS4 | YP_003969055.1 |
| Aeromonas | Aeromonas phage 44RR2.8t | NP_932578.1 |
| Aeromonas | Aeromonas phage 25 | YP_656449.1 |
| Aeromonas | Aeromonas phage 31 | YP_238949.1 |
| Aeromonas | Aeromonas phage Aeh1 | NP_944217.1 |
| Aeromonas | Aeromonas phage 65 | YP_004300997.1 |
| Aeromonas | Aeromonas phage phiAS5 | YP_003969406.1 |
| Escherichia | Escherichia phage wV7 | AEM00790.1 |
| Escherichia | Enterobacteria phage T4 | NP_049736.1 |
| Escherichia | Enterobacteria phage vB_EcoM-VR7 | YP_004063811.1 |
| Escherichia | Enterobacteria phage Bp7 | AEN93735.1 |
| Escherichia | Enterobacteria phage JS98 | YP_001595245.1 |
| Escherichia | Enterobacteria phage AR1 | BAI83135.1 |
| Escherichia | Enterobacteria phage JS10 | YP_002922463.1 |
| Escherichia | Enterobacteria phage IME08 | YP_003734260.1 |
| Escherichia | Enterobacteria phage CC31 | YP_004009990.1 |
| Escherichia | Enterobacteria phage RB69 | NP_861818.1 |
| Escherichia | Enterobacteria phage RB14 | YP_002854463.1 |
| Escherichia | Enterobacteria phage RB32 | ABI94948.1 |
| Escherichia | Enterobacteria phage RB51 | YP_002854084.1 |
| Shigella | Shigella phage Shfl2 | YP_004415022.1 |

The sequences of the endolysins of table 1 may be accessed for instance via the protein database of the National Center for Biotechnology Information (NCBI; https://www.ncbi.nlm.nih.gov/). It is understood that the sequences of the endolysins listed in table 1 may also be modified, e.g. may lack the N-terminal methionine to avoid a further start codon in the corresponding nucleic acid sequence. Using such marginally amended sequences is also within the scope of the present invention and it is understood, that when reference herein is made to endolysins of table 1, that also such modified endolysins are encompassed by said definition. Furthermore, the inventors envisage that also endolysins exhibiting at least 80% sequence identity, preferably at least 85% sequence identity, more preferably at least 90% sequence identity, more preferably at least 95% sequence identity, more preferably at least 96% sequence identity, more preferably at least 97% sequence identity, more preferably at least 98% sequence identity, more preferably at least 99% sequence identity with a sequence of table 1 may also be used for carrying out the present invention.

It is understood that any sequence discussed herein as sharing a level of sequence identity, e.g. with SEQ ID NO:21, SEQ ID NO:22, SEQ ID NO:27, SEQ ID NO:28 or SEQ ID NO:30, respectively, need to retain the sequence according to SEQ ID NO:1 (or SEQ ID NO:2 or SEQ ID NO:7), i.e. the deviation in sequence will be outside SEQ ID NO:1 (or SEQ ID NO:2 or SEQ ID NO:7, respectively), not within the sequence corresponding to the motif of SEQ ID NO:1 or SEQ ID NO:2 or SEQ ID NO:7, respectively).

As mentioned previously, it is meanwhile established for about a decade that Gram negative bacteria can be killed with respective Gram negative endolysins even if added from without, if the endolysins are fused with, e.g., an antimicrobial peptide, an amphipathic peptides or a cationic peptide (see for example WO 2010/023207, WO 2010/149792, WO 2011/134998, WO 2012/146738, or WO 2015/121443, all incorporated herein by reference). In the following, this peptide within the inventive polypeptide will also be referred to as “peptide component”. It is understood that this peptide component, as used herein, is not a conventional tag like His-tags, such as HisS-tags, His6-tags, His7-tags, His8-tags, His9-tags, His10-tags, His11-tags, His12-tags, His16-tags and His20-tags, Strep-tags, Avi-tags, Myc-tags, Gst-tags, JS-tags, cystein-tags, FLAG-tags or other tags known in the art, such as thioredoxin or maltose binding proteins (MBP).

Preferred cationic and/or polycationic peptides are those comprising at least one motive according to SEQ ID NO:37 (KRKKRK). In particular cationic amino acid sequence stretches comprising at least 2, 3, 4, 5, 6, 7, 8, 9, 10, 11, 12, 13, 14, 15, 16 or 17 motives according to SEQ ID NO: 37 (KRKKRK) are preferred. More preferred are cationic peptide stretches comprising at least one KRK motive (lys-arg-lys), preferable at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 KRK motives.

In another preferred embodiment of the present invention the cationic peptide comprises beside the positively charged amino acid residues, in particular lysine and/or arginine residues, neutrally charged amino acid residues, in particular glycine and/or serine residues. Preferred are cationic amino acid sequence stretches consisting of about 70% to about 100%, or about 80% to about 95%, or about 85% to about 90% positively charged amino acid residues, in particular lysine, arginine and/or histidine residues, more preferably lysine and/or arginine residues and of about 0% to about 30%, or about 5% to about 20%, or about 10% to about 20% neutrally charged amino acid residues, in particular glycine and/or serine residues. Preferred are amino acid sequence stretches consisting of about 4% to about 8% serine residues, of about 33% to about 36% arginine residues and of about 56% to about 63% lysine residues. Especially preferred are amino acid sequence stretches comprising at least one motive according to SEQ ID NO: 38 (KRXKR), wherein X is any other amino acid than lysine, arginine and histidine. Especially preferred are polypeptide stretches comprising at least one motive according to SEQ ID NO: 39 (KRSKR). More preferred are cationic stretches comprising at least about 2, 3, 4, 5, 6, 7, 8, 9, 10, 11, 12, 13, 14, 15, 16, 17, 18, 19 or at least about 20 motives according to SEQ ID NO: 38 (KRXKR) or SEQ ID NO: 39 (KRSKR).

Also preferred are cationic amino acid sequence stretches consisting of about 9 to about 16% glycine residues, of about 4 to about 11% serine residues, of about 26 to about 32% arginine residues and of about 47 to about 55% lysine residues. Especially preferred are amino acid sequence stretches comprising at least one motive according to SEQ ID NO: 40 (KRGSG). More preferred are cationic stretches comprising at least about 2, 3, 4, 5, 6, 7, 8, 9, 10, 11, 12, 13, 14, 15, 16, 17, 18, 19 or at least bout 20 motives according to SEQ ID NO: 40 (KRGSG).

In another preferred embodiment of the present invention such cationic amino acid sequence stretch comprises beside the positively charged amino acid residues, in particular lysine and/or arginine residues, hydrophobic amino acid residues, in particular valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues. Preferred are cationic amino acid sequence stretches consisting of about 70% to about 100%, or about 80% to about 95%, or about 85% to about 90% positively charged amino acid residues, in particular lysine and/or arginine residues and of about 0% to about 30%, or about 5% to about 20%, or about 10% to about 20% hydrophobic amino acid residues, valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues. Examples for cationic and polycationic amino acid sequence stretches are listed in the following table:

TABLE 2

| amino acid | | SEQ ID |
|---|---|---|
| sequence stretch | length | NO: |
| KRKKRK | 6 | 37 |
| KRKKRKKRK | 9 | 41 |
| RRRRRRRRR | 9 | 42 |
| KKKKKKKK | 8 | 43 |
| KRKKRKKRKK | 10 | 44 |
| KRKKRKKRKKRK | 12 | 45 |
| KRKKRKKRKKRKKR | 14 | 46 |
| KKKKKKKKKKKKKKKK | 16 | 47 |
| KRKKRKKRKKRKKRKK | 18 | 48 |
| RK | | |
| KRKKRKKRKKRKKRKK | 19 | 49 |
| RKK | | |
| RRRRRRRRRRRRRRRR | 19 | 50 |
| RRR | | |
| KKKKKKKKKKKKKKKK | 19 | 51 |
| KKK | | |
| KRKKRKKRKRSKRKKR | 20 | 52 |
| KKRK | | |
| KRKKRKKRKRSKRKKR | 21 | 53 |
| KKRKK | | |
| KRKKRKKRKKRKKRKK | 21 | 54 |
| RKKRK | | |
| KRKKRKKRKRGSGKRK | 22 | 55 |
| KRKKRK | | |
| KRKKRKKRKRGSGSGK | 24 | 56 |
| RKKRKKRK | | |
| KRKKRKKRKKRKKRKK | 25 | 57 |
| RKKRKKRKK | | |
| KRKKRKKRKRSKRKKR | 31 | 58 |
| KKRKRSKRKKRKKRK | | |
| KRKKRKKRKRGSGSGK | 38 | 59 |
| RKKRKKRKGSGSGKRK | | |
| KRKKRK | | |
| KRKKRKKRKKRKKRKK | 39 | 60 |
| RKKRKKRKKRKKRKKR | | |
| KKRKKRK | | |
| KRKKRKKRKRSKRKKR | 42 | 61 |
| KKRKRSKRKKRKKRKR | | |
| SKRKKRKKRK | | |

In a further aspect of the present invention the peptide is an antimicrobial peptide, which comprises a positive net charge and around 50% hydrophobic amino acids. The antimicrobial peptides are amphipathic with a length of about 12 to about 50 amino acid residues. The antimicrobial peptides are naturally occurring in insects, fish, plants, arachnids, vertebrates or mammals. Preferably the antimicrobial peptide may be naturally occurring in radish, silk moth, wolf spider, frog, preferably in Xenopus laevis, Rana frogs, more preferably in Rana catesbeiana, toad, preferably Asian toad Bufo bufo gargarizans, fly, preferably in Drosophila, more preferably in Drosophila melanogaster, in Aedes aegypti, in honey bee, bumblebee, preferably in Bombus pascuorum, flesh fly, preferably in Sarcophaga peregrine, scorpion, horseshoe crab, catfish, preferably in Parasilurus asotus, cow, pig, sheep, porcine, bovine, monkey and human.

In another preferred embodiment of the present invention the antimicrobial peptide consists of about 10% to about 35% or about 15% to about 45%, or about 20% to about 45% positively charged amino acid residues, in particular lysine and/or arginine residues and of about 50% to about 80%, or about 60% to about 80%, or about 55% to about 75%, or about 70% to about 90% hydrophobic amino acid residues, valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues.

In another preferred embodiment of the present invention the antimicrobial peptide consist of about 4% to about 58% positively charged amino acid residues, in particular lysine and/or arginine residues and of about 33% to about 89% hydrophobic amino acid residues, valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues.

Examples for antimicrobial amino acid sequences which may be used in carrying out the present invention are listed in the following table.

TABLE 3

| | | SEQ |
|---|---|---|
| | | ID |
| Peptide | Sequence | NO |
| LL-37 | LLGDFFRKSKEKIGKE | 62 |
| | FKRIVQRIKDFLRNLV | |
| | PRTES | |
| SMAP-29 | RGLRRLGRKJAHGVKK | 63 |
| | YGPTVLRIIRIAG | |
| Indolicidin | ILPWKWPWWPWRR | 64 |
| Protegrin | RGGRLCYCRRRFCVCV | 65 |
| | GR | |
| Cecropin P1 | SWLSKTAKJCLENSAK | 66 |
| | KRISEGIAIAIQGGPR | |
| Magainin | GIGKFLHSAKKFGKAF | 67 |
| | VGEIMNS | |
| Pleurocidin | GWGSFFKKAAHVGKHV | 68 |
| | GKAALTHYL | |
| Cecropin A | GGLKKLGKKLEGAGKR | 69 |
| (A.aegypti) | VFNAAEKALPVVAGAK | |
| | ALRK | |
| Cecropin A (D. | GWLKKIGKKIERVGQH | 70 |
| melanogaster) | TRDATIQGLGIPQQA | |
| | ANVAATARG | |
| Buforin II | TRSSRAGLQFPVGRVH | 71 |
| | RLLRK | |
| Sarcotoxin IA | GWLKJCIGKKIERVGQ | 72 |
| | HTRDATIQGLGIAQQA | |
| | ANVAATAR | |
| Apidaecin | ANRPVYIPPPRPPHPR | 73 |
| | L | |
| Ascaphine 5 | GIKDWIKGAAKKLIKT | 74 |
| | VASHIANQ | |
| Nigrocine 2 | GLLSKVLGVGKKVLCG | 75 |
| | VSGLVC | |
| Pseudin 1 | GLNTLKKVFQGLHEAI | 76 |
| | KLINNHVQ | |
| Ranalexin | FLGGLIVPAMICAVTK | 77 |
| | KC | |
| Melittin | GIGAVLKVLTTGLPAL | 78 |
| | ISWIKRKRQQ | |
| Lycotoxin 1 | IWLTALKFLGKHAAKK | 79 |
| | LAKQQLSKL | |
| Parasin 1 | KGRGKQGGKVRAKAKT | 80 |
| | RSS | |
| Buforin I | AGRGKQGGKVRAKAKT | 81 |
| | RSSRAGLQFPVGRVHR | |
| | LLRKGNY | |
| Dermaseptin 1 | ALWKTMLKKLGTMALHAG | 82 |
| | KAALGAAADTISQGTQ | |
| Bactenecin 1 | RLCRIVVIRVCR | 83 |
| Thanatin | GSKKPVPIIYCNRRTG | 84 |
| | KCQRM | |
| Brevinin IT | VNPIILGVLPKVCLIT | 85 |
| | KKC | |
| Ranateurin 1 | SMLSVLKNLGKVGLGF | 86 |
| | VACKINIKQC | |
| Esculentin 1 | GIFSKLGRKKIKNLLI | 87 |
| | SGLKNVGKEVGMDVVR | |
| | TGIKIAGCKIKGEC | |
| Tachyplesin | RWCFRVCYRGICYRKC | 88 |
| | R | |
| Androctonin | RSVCRQIKICRRRGGC | 89 |
| | YYKCTNRPY | |
| alpha- | DCYCRIPACIAGERRY | 90 |
| defensin | GTCIYQGRLWAFCC | |
| beta- | NPVSCVRNKGICVPIR | 91 |
| defensin | CPGSMKQIGTCVGRA | |
| | VKCCRKK | |
| theta- | GFCRCLCRRGVCRCIC | 92 |
| defensin | TR | |
| defensin | ATCDLLSGTGINHSAC | 93 |
| | AAHCLLRGNRGGYCN | |
| (sapecin A) | GKAVCVCRN | |
| Thionin | TTCCPSIVARSNFNVC | 94 |
| (crambin) | RIPGTPEAICATYTGC | |
| | IIIPGATCPGDYAN | |
| Defensin | QKLCQRPSGTWSGVC | 95 |
| from | GNNNACKNQCIRLEK | |
| radish | ARHGSCNYVFPAHCI | |
| | CYFPC | |
| Cathelecidin- | KFFRKLKKSVKKRAK | 96 |
| BF | EFFKKPRVIGVSIPF | |
| Drosomycin | DCLSGRYKGPCAVWD | 97 |
| | NETCRRVCKEEGRSS | |
| | GHCSPSLKCWCEGC | |
| Hepcidin | DTHFPICIFCCGCCH | 98 |
| | RSKCGMCCKT | |
| Bac 5 | RFRPPIRRPPIRPPF | 99 |
| | YPPFRPPIRPPIFPP | |
| | IRPPFRPPLGRPFP | |
| PR-39 | RRRPRPPYLPRPRPP | 100 |
| | PFFPPRLPPRIPPGF | |
| | PPRFPPRFP | |
| Pyrrhocoricin | VDKGSYLPRPTPPRP | 101 |
| | IYNRN | |
| Histatin 5 | DSHAKRHHGYKRKFH | 102 |
| | EKHHSHRGY | |
| ECP19 | RPPQFTRAQWFAIQH | 103 |
| | ISLN | |
| MSI-594 | GIGKFLKKAKKGIGA | 104 |
| | VLKVLTTG | |
| TL-ColM | METLTVHAPSPSTNL | 105 |
| | PSYGNGAFSLSAPHV | |
| | PGAGP | |
| SBO | KLKKIAQKIKNFFAK | 106 |
| | LVA | |

A particularly preferred antimicrobial peptide for use in the inventive polypeptide of the present invention is SMAP-29 (SEQ ID NO:63).

Further particularly preferred antimicrobial peptides are peptides according to SEQ ID NO: 96 and SEQ ID NO:107.

In a further embodiment the peptide component of the inventive polypeptide may be a sushi peptide which is described by Ding J L, Li P, Ho B Cell Mol Life Sci. 2008 April; 65(7-8):1202-19. The Sushi peptides: structural characterization and mode of action against Gram-negative bacteria. Especially preferred is the sushi 1 peptide according to SEQ ID NO: 108. Preferred sushi peptides are sushi peptides S1 and S3 and multiples thereof; Tan et al. FASEB J. 2000 September; 14(12):1801-13.

In a further aspect of the present invention the peptide component is an amphipathic peptide, which comprises one or more of the positively charged amino acid residues of lysine, arginine and/or histidine, combined to one or more of the hydrophobic amino acid residues of valine, isoleucine, leucine, methionine, phenylalanine, tryptophan, cysteine, alanine, tyrosine, proline and/or glycine. Side chains of the amino acid residues are oriented in order that cationic and hydrophobic surfaces are clustered at opposite sides of the peptide. Preferably, more than about 30, 40, 50, 60 or 70% of the amino acids in said peptide are positively charged amino acids. Preferably, more than about 30, 40, 50, 60 or 70%, of the amino acid residues in said peptide are hydrophobic amino acid residues. Advantageously, the amphipathic peptide is present at the N-terminal (most preferred) or the C-terminal end of the polypeptide according to the present invention.

In another embodiment of the invention, the amphipathic peptide consists of at least 5, more preferably at least of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 amino acid residues. In a preferred embodiment at least about 30, 40, 50, 60 or 70% of said amino acid residues of the amphipathic peptide are either arginine or lysine residues and/or at least about 30, 40, 50, 60 or 70% of said amino acid residues of the amphipathic peptide are of the hydrophobic amino acids valine, isoleucine, leucine, methionine, phenylalanine, tryptophan, cysteine, alanine, tyrosine, proline and/or glycine.

In another preferred embodiment of the present invention the amphipathic peptide stretch comprises beside the positively charged amino acid residues, in particular lysine and/or arginine residues, hydrophobic amino acid residues, in particular valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues. Preferred are amphipathic peptide stretches consisting of about 10% to about 50%, or about 20% to about 50%, or about 30% to about 45% or about 5% to about 30% positively charged amino acid residues, in particular lysine and/or arginine residues and of about 50% to about 85%, or about 50% to about 90%, or about 55% to about 90%, or about 60% to about 90%, or about 65% to about 90% hydrophobic amino acid residues, valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues. In another preferred embodiment amphipathic peptide stretches consisting of 12% to about 50% positively charged amino acid residues, in particular lysine and/or arginine residues and of about 50% to about 85% hydrophobic amino acid residues, valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues.

Preferred amphipathic peptides are WLBU2-Variant having the amino acid sequence according to SEQ ID NO: 109 and Walmagh 2 according to SEQ ID NO: 110.

In a particularly preferred embodiment of the present invention, the peptide (within the inventive polypeptide) comprises a sequence motif which:

i) is 16, 17, 18, 19 or 20 amino acids in length;

ii) comprises at least 40% and at most 60% amino acids selected from a first group of amino acids consisting of lysine, arginine and histidine,
wherein each amino acid is selected independently from said first group,
wherein each amino acid selected from this first group is arranged in said sequence motif either alone, pairwise together with a further amino acid selected from the first group, or in a block with 2 further amino acids selected from the first group, but does not occur in a block with 3 or more amino acids selected from the first group, wherein at least 2 pairs of amino acids selected from the first group are present in said sequence motif, and wherein at most one block with 3 of the amino acids selected from the first group in a row is present in said sequence motif, with the additional proviso, that if such block with 3 amino acids of the first group is present in said sequence motif, then the amino acids at positions 12, −11, −8, −5, −4, +6, +7, +10, +13, and +14 relative to the first amino acid of the 3 amino acid block are—provided the respective position may be found in said sequence motif—not selected from said first group,

iii) comprises at least 40% and at most 60% amino acids selected from a second group of amino acids consisting of alanine, glycine, isoleucine, leucine, phenylalanine, serine, threonine, tryptophan, tyrosine and valine,
wherein each amino acid is selected independently from said second group,
wherein preferably at least three different amino acids are selected from this second group, if the sum of amino acids selected from the first group and selected from the second group yield 100% of the sequence motif;
wherein preferably the sequence motif does not comprise the sequence AFV, if the sequence motif contains at least two single, non-adjacent phenylalanine residues and at least one of these phenylalanine residues is directly preceded by a lysine residue, and
wherein the sequence motif does preferably not comprise the sequence AALTH (SEQ ID NO:111), if the sequence motif contains at least three non-adjacent histidine residues,

iv) wherein the remaining amino acids of said sequence motif, if any are present in the motif, are selected from a third group consisting of asparagine, aspartic acid, glutamine, glutamic acid, methionine, or cysteine, wherein each of said amino acids is selected independently from said third group, and wherein glutamine may be preferably selected only once and wherein the selection may preferably furthermore not comprise a combination of glutamine and glutamic acid.

The sequence motif defined above in i) to iv) may represent only a part of the peptide component of the inventive polypeptide, i.e. the peptide component of the inventive polypeptide is longer than the sequence motif Alternatively, the sequence motif may be the sequence of the peptide component, i.e. the sequence of the peptide component in the inventive polypeptide is identical to the sequence of the sequence motif Moreover, and as will be apparent from the example provided in FIG. 2, it is possible that the inventive polypeptide comprises one or more such sequence motifs. For instance, the 20mer motif may inherently comprise a 16mer motif also complying with the criteria set out above. The fact, that the peptide component of the inventive polypeptide comprises “a” sequence motif as defined above may thus not be understood as meaning that the inventive polypeptide may only comprise “one” sequence motif and no further (e.g. overlapping) sequence motifs also complying with the limits set out above.

The sequence motif of the peptide component of the inventive polypeptide may be 16, 17, 18, 19 or 20 amino acids in length. Preferably, the sequence motif is 17, 18 or 19 amino acids in length, even more preferably 17 or 18 amino acids in length.

The sequence motif of the peptide component of the inventive polypeptide comprises at least 40% and at most 60% amino acids selected from a first group of amino acids. Said first group consists of lysine, arginine and histidine. If the sequence motif is 16 amino acids long, it will exhibit at least 7 and at most 9 amino acids selected from this first group. If the sequence motif is 17 amino acids long, it will exhibit at least 7 and at most 10 amino acids selected from this first group. If the sequence motif is 18 amino acids long, it will exhibit at least 8 and at most 10 amino acids selected from this first group. If the sequence motif is 19 amino acids long, it will exhibit at least 8 and at most 11 amino acids selected from this first group. If the sequence motif is 20 amino acids long, it will exhibit at least 8 and at most 12 amino acids selected from this first group.

Preferred amino acids for selection within this first group are lysine and arginine. Preferably, the sequence motif does not comprise more than 50% histidine residues. Even more preferably, the sequence motif does not comprise more than 25% histidine residues. In some embodiments of the invention, the sequence motif comprises only one or even no histidine residue.

The amino acids selected from the first group are selected independently. This implies, for example, that if a given sequence motif comprises, e.g., eight amino acids selected from the first group, that each of these eight amino acid residues can be selected independently from previous or subsequent selections from said first group. The selected amino acids may thus comprise all three types of amino acids (lysine, arginine, and histidine), may be identical (e.g. 8 lysine or 8 arginine residues, respectively), or may comprise only two of the three types of amino acids (e.g. lysine and arginine). Likewise, independent selection does not prescribe any specific ratio between the individually selected amino acids. For example, and without being limited thereto, 8 amino acids selected from this first group may be 8 lysine residues, 7 arginine residues and 1 histidine residue or 3 arginine, 4 lysine and 1 histidine residue.

The positioning of the amino acid residues selected from the first group within the sequence motif is subject to certain limitations. Each amino acid selected from this first group may only be arranged in said sequence motif either alone, pairwise together with a further amino acid selected from the first group, or in a block with 2 further amino acids selected from the first group.

“Alone” means that an amino acid selected from said first group, e.g. lysine (K), is neither N-terminally nor C-terminally flanked by another amino acid from said first group. Adjacent amino acid residues may be selected from the second or, as the case may be, from the third group (e.g. LKE, N-KE (at N-terminus of motif), LK-C (at C-terminus of motif)). Noteworthy, potential further amino acids within the inventive polypeptide, but outside of the sequence motif, are not taken into account for this positional determination. An amino acid from the first group at one of the two ends of the sequence motif is thus considered to be positioned alone, even if the preceding (N-terminus) or subsequent (C-terminus) amino acid residue outside of the sequence motif is by chance also an arginine, histidine or lysine residue.

“Pairwise together with a further amino acid selected from the first group” means that within the sequence motif an amino acid selected from the first group is directly adjacent to another amino acid selected from the first group. This two amino acids form thereby a pair of amino acids selected from the first group. Said pair in turn is flanked C-terminally and N-terminally by amino acids from the second or, as the case may be, from the third group (e.g., LKRE (SEQ ID NO:112), N-KRE (at N-terminus of motif), LKR-C (at C-terminus of motif)). Potential further amino acids within the peptide component of the inventive polypeptide, but outside of the sequence motif, are again not taken into account for this positional determination.

“In a block with 2 further amino acids selected from the first group” means that three amino acids selected from the first group are directly adjacent to each other. Said block (or triplet) is flanked C-terminally and N-terminally by amino acids from the second or, as the case may be, from the third group (e.g., LKRKE (SEQ ID NO:113), N-KRKE (at N-terminus of motif; SEQ ID NO:114), LKRK-C (at C-terminus of motif; SEQ ID NO:115)). Potential further amino acids within the peptide component of the inventive polypeptide, but outside of the sequence motif, are again not taken into account for this positional determination. For amino acids arranged in such manner (triplet; block with 3 amino acids of the first group) an additional positional requirement must be met, namely that none of the amino acids at positions −12, −11, −8, −5, −4, +6, +7, +10, +13, and +14 relative to the first amino acid of the 3 amino acid block is—provided the respective position may be found in said sequence motif—an amino acid selected from said first group. Negative values indicate positions N-terminal of the first amino acid of the triplet; positive values refer to positions C-terminal of the first amino acid of the triplet. Basis for the positional calculation is the first (N-terminal) amino acid of the triplet (e.g. the amino acid directly N-terminal of the triplet would be −1, the amino acid directly C-terminal of the triplet would be +3). This limitation thus precludes a sequence like RRRGLRH (SEQ ID NO:116), because position +6 (H) is an amino acid of the first group. Whether the respective positions (−12, −11, −8, −5, −4, +6, +7, +10, +13, and +14) are present in the sequence motif or not will be dependent on the position of the triplet within the sequence motif and the length of the sequence motif. For example, if the triplet would be situated at the N-terminus of the sequence motif, then all negative values are obsolete (i.e. need not be taken into account). The same applies for the positive values, if the triplet is situated at the C-terminus of the sequence motif. However, in preferred embodiments, the sequence motif does not comprise such triplet block of amino acids of the first group at all, i.e. does not comprise a block consisting of 3 amino acids selected from the first group.

It is understood that the positional requirements alone, pairwise together with a further amino acid selected from the first group, and in a block with 2 further amino acids selected from the first group are not overlapping and the terms are mutual exclusive (e.g. a triplet is not a case of “alone” and/or “pairwise together”, etc.).

A further positional requirement for the amino acids selected from the first group is, that the sequence motif must comprise at least 2 pairs of amino acids selected from the first group. However, it is preferred that not all amino acids selected from the first group are arranged pairwise in the sequence motif.

The sequence motif of the inventive polypeptide does not comprise blocks of 4 (quartet) or more amino acids (quintet, sextet, etc.) selected from the first group (i.e. an amino acid of the first group does not occur in a block with 3 or more amino acids selected from the first group). The sequence motif may thus for example not comprise sequences such as “KRKK” (SEQ ID NO:117) or “RRRR” (SEQ ID NO:118).

As amino acids of the first group make up only 40% to 60% of the sequence motif, the remaining amino acids need to be selected from other amino acid residues. As set out above, the sequence motif comprises also at least 40% and at most 60% amino acids selected from a second group of amino acids. Said second group consists of the amino acid residues alanine, glycine, isoleucine, leucine, phenylalanine, serine, threonine, tryptophan, tyrosine and valine. As before for the first group of amino acids, each of the amino acids of the second group is likewise in principle selected independently, i.e. each amino acid is selected independent from any previous or subsequent selections from said second group.

However, for the second group there are preferably some restrictions to this general principle of independent selection. The first restriction preferably applies, if the sum of amino acids selected from the first group and selected from the second group yields 100% of the amino acids of the sequence motif (i.e. there are no amino acids from the third group in the sequence motif). In such scenario at least three different amino acids are preferably selected from the second group. In such scenario the amino acids of the second group may for example preferably not be restricted to valine and tryptophan residues only.

A further preferred (positional) restriction is that the sequence motif may not comprise the triplet sequence AFV (alanine, phenylalanine, valine), if the sequence motif contains at least two single, non-adjacent phenylalanine residues and at least one of these phenylalanine residues is (N-terminally) directly preceded by a lysine residue (i.e. KF). Nonadjacent phenylalanine residues are phenylalanine residues which do not occur in a row in the sequence, but which are separated by one or more other amino acids. Single phenylalanine residues means that they are not part of a pair of phenylalanine residues or of a block of several phenylalanine residues but are positioned alone in the sequence motif.

The next preferred restriction is, that the sequence motif does not comprise the sequence AALTH (i.e. alanine, alanine, lysine, threonine, histidine), if the sequence motif contains at least three single, non-adjacent histidine residues. Nonadjacent histidine residues are histidine residues which do not occur in a row, but which are separated by one or more other amino acids. Single histidine residues means that they are not part of a pair of histidine residues or of a block of several histidine residues but are positioned alone in the sequence motif.

In a preferred embodiment, less than 5 isoleucine residues (e.g. 4, 3, 2, 1 or 0) are selected from said second group.

It is possible, that the sequence motif of the peptide component of the polypeptide of the invention is not exclusively composed of amino acids selected from the first and second group (i.e. they represent together less than 100%). In such scenario, the remaining amino acids of said sequence motif are selected from a third group of amino acids, said group consisting of asparagine, aspartic acid, glutamine, glutamic acid, methionine, and cysteine. As before for the first and second group of amino acids, each of the amino acids of the third group is likewise in principle selected independently, i.e. each amino acid is selected independent from any previous or subsequent selections from said third group. However, as before for the second group, there are some preferred restrictions to the selection of an amino acid from said third group: glutamine may be selected only once and a selection of glutamine and glutamic acid in parallel is also not allowed, i.e. if glutamine is present in the sequence motif, then no glutamic acid may be present and vice versa). Preferably, the amino acids selected from the third group are limited to asparagine, aspartic acid, glutamine and glutamic acid, i.e. the selected third group amino acids do not comprise methionine or cysteine residues.

In preferred embodiments, the sequence motif comprises only a single, or even more preferred no amino acid residue at all from the third group.

In preferred embodiments of the present invention, the arrangement of the selected amino acids in the sequence motif complies with the requirements set out in one of the possible sequence motif alternatives depicted in FIG. 2. FIG. 2 specifies that at specific positions for a given 16mer, 17mer, 18mer, 19mer or 20mer no amino acids selected from the first group may be present. At these positions only amino acids selected from the second and/or the third group (if any) may be present. Preferably, amino acids of the second group are present at said positions. Amino acids of the first group may only be present at any of the remaining positions of the sequence motif This does not imply that at these remaining positions only amino acids of the first group may be found. Amino acids of the second and optionally third group may also be found at these remaining positions, provided the overall percentage requirements for the first and second group are still met.

Preferably, the sequence motif of the peptide component is of helical structure.

The preferred sequence motif of the peptide component does not comprise any other amino acid residues than those defined to be in the first, second or third group. In particular, the preferred sequence motif of the peptide component does not comprise any proline residue, and if the third group is limited to asparagine, aspartic acid, glutamine and glutamic acid, no methionine and cysteine as well.

However, a proline residue may very well be present elsewhere in the peptide component (or inventive polypeptide). It is for example preferred, if a proline residue is located within 1 to 10, preferably 1 to 5 amino acid residues N-terminal or C-terminal of the sequence motif, with the latter being preferred. It is furthermore preferred if such proline residue is found between the sequence of the endolysin and the sequence motif Preferably, the sequence motif is N-terminal of the sequence of the endolysin and the proline residue is positioned somewhere in between, usually close to the sequence motif.

Examples for peptide components exhibiting the above discussed preferred sequence motif are peptides comprising the sequence of SEQ ID NO:63, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID

NO:135 and SEQ ID NO:136. A particularly preferred peptide component exhibiting the above mentioned sequence motif is SEQ ID NO:132.

The peptide (component) of the inventive polypeptide consists preferably of at least 5, more preferably at least of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 amino acid residues. Especially preferred are those peptides consisting of about 5 to about 100 amino acid residues, about 5 to about 50 or about 5 to about 30 amino acid residues. More preferred are peptide stretches consisting of about 6 to about 42 amino acid residues, about 6 to about 39 amino acid residues, about 6 to about 38 amino acid residues, about 6 to about 31 amino acid residues, about 6 to about 25 amino acid residues, about 6 to about 24 amino acid residues, about 6 to about 22 amino acid residues, about 6 to about 21 amino acid residues, about 6 to about 20 amino acid residues, about 6 to about 19 amino acid residues, about 6 to about 16 amino acid residues, about 6 to about 14 amino acid residues, about 6 to about 12 amino acid residues, about 6 to about 10 amino acid residues or about 6 to about 9 amino acid residues.

In a preferred embodiment the inventive polypeptide comprises at least one amino acid sequence selected from the group consisting of KRK and SEQ ID NOs: 37-136.

The peptide component of the polypeptide according to the present invention may be linked to the endolysin by intervening additional amino acid residues e.g. due to cloning reasons. Alternatively, the peptide component may be directly linked to the endolysin sequence without any intervening linker sequences.

Preferably, said intervening additional amino acid residues may not be recognized and/or cleaved by proteases. Preferably said additional amino acid sequences are linked to each other and/or to the enzyme by at least 1, 2, 3, 4, 5, 6, 7, 8, 9 or 10 additional intervening amino acid residues.

In a preferred embodiment the peptide is linked to the rest of the inventive polypeptide, preferably at the N- or C-terminus of the polypeptide according to the present invention, by the additional intervening amino acid residues glycine, serine and serine (Gly-Ser-Ser), glycine, alanine, glycine and alanine (Gly-Ala-Gly-Ala; SEQ ID NO:137), glycine, alanine, glycine, alanine, glycine, alanine, glycine and alanine (Gly-Ala-Gly-Ala-Gly-Ala-Gly-Ala; SEQ ID NO:138) or glycine, alanine, glycine, alanine, glycine, alanine, glycine, alanine, glycine, alanine, glycine and alanine (Gly-Ala-Gly-Ala-Gly-Ala-Gly-Ala-Gly-Ala-Gly-Ala; SEQ ID NO:139).

Preferably, the peptide component is situated N-terminal of the endolysin within the inventive polypeptide. In such scenario (in particular for the fifth and sixth aspect of the polypeptide of the present invention, but not limited thereto) it is particularly preferred that the endolysin constitutes the most C-terminal component of the polypeptide, i.e. there are no further functional elements C-terminal of the endolysin sequence. Preferably, there are 10 or less, more preferably 5 or less, more preferably 4 or less, more preferably 3 or less, more preferably 2 or less, more preferably only 1 and most preferably no amino acids C-terminal of the endolysin sequence in an inventive polypeptide.

Examples of polypeptides of the present invention are polypeptides comprising for instance as an endolysin SEQ ID NO:28 or SEQ ID NO:30 (or a sequence sharing at least 80% sequence identity therewith) and further comprising as peptide component SEQ ID NO:63 or SEQ ID NO:132. Particularly preferred polypeptides according to the present invention are polypeptides comprising SEQ ID NO:140 or SEQ ID NO:141 and polypeptides sharing at least 80% sequence identity, preferably at least 85% sequence identity, more preferably at least 90% sequence identity, more preferably at least 95% sequence identity, more preferably at least 96% sequence identity, more preferably at least 97% sequence identity, more preferably at least 98% sequence identity, more preferably at least 99% sequence identity with SEQ ID NO:140 and/or SEQ ID NO:141.

Other examples of polypeptides according to the present invention are polypeptides comprising as endolysin component for example SEQ ID NO:31, SEQ ID NO:32, SEQ ID NO:33, SEQ ID NO:34, SEQ ID NO:35, or SEQ ID NO:36 (or a sequence sharing at least 80% sequence identity with any of these), and as peptide component selected from the group consisting of SEQ ID NO: 96, SEQ ID NO: 107, SEQ ID NO:132, SEQ ID NO: 133, SEQ ID NO: 134, SEQ ID NO: 135 or SEQ ID NO: 136. Examples for such inventive polypeptides are provided as SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151 and SEQ ID NO:152, as well as polypeptides sharing at least 80% sequence identity, preferably at least 85% sequence identity, more preferably at least 90% sequence identity, more preferably at least 95% sequence identity, more preferably at least 96% sequence identity, more preferably at least 97% sequence identity, more preferably at least 98% sequence identity, more preferably at least 99% sequence identity with any of these sequences.

SEQ ID NO:140, SEQ ID NO:141, SEQ ID NO:143, SEQ ID NO:144, SEQ ID NO:145, SEQ ID NO:146, SEQ ID NO:147, SEQ ID NO:148, SEQ ID NO:151 and SEQ ID NO:152 exhibit an alanine residue at the N-terminus (instead of, e.g., a methionine residue. Said alanine residue is not critical and merely remained after proteolytic removal of a His-Tag at the N-terminus. In the case of SEQ ID NO:115 proteolytic removal of the His-Tag at the N-terminus left no additional amino acid, i.e. the polypeptide directly starts with the peptide according to SEQ ID NO:106.

Aside of the endolysin and peptide, as defined herein, the inventive polypeptide may of course also comprise other amino acid sequence elements, e.g. one or more tags, e.g. a His-tag, Strep-tag, Avi-tag, Myc-tag, Gst-tag, JS-tag, cystein-tag, FLAG-tag or other tags known in the art, thioredoxin, maltose binding proteins (MBP) etc.

In this context, the inventive polypeptide may additional comprise a tag e.g. for purification. Preferred is a His6-tag (SEQ ID NO: 153), preferably at the C-terminus and/or the N-terminus of the polypeptide according to the present invention. Said tag can be linked to the polypeptide by additional amino acid residues e.g. due to cloning reasons. Preferably said tag can be linked to the protein by at least 1, 2, 3, 4, 5, 6, 7, 8, 9 or 10 additional amino acid residues. Preferably said additional amino acid residues may be recognized and/or cleaved by proteases. In a preferred embodiment the inventive polypeptide comprises a His6-tag at its N-terminus.

In a seventh aspect the present invention relates to a polypeptide comprising the sequence of a peptide selected from the group consisting of SEQ ID NO: 107, SEQ ID NO:133, SEQ ID NO:134, SEQ ID NO:135 and SEQ ID NO: 136 and optionally the sequence of a muralytic enzyme. The muralytic enzyme of the polypeptide according to the seventh aspect of the invention may be any muralytic enzyme (in particular peptidoglycan hydrolase) capable of degrading bacterial peptidoglycan. Such muralytic enzyme may be in terms of enzymatic activity for example an endopeptidase, N-acetyl-muramoyl-L-alanine-amidase (amidase), N-acetyl-muramidase, N-acetyl-glucosaminidase or lytic transglycosylase and is thus suitable for degrading the peptidoglycan of bacterial cell walls. Preferably, the muralytic enzyme degrades the peptidoglycan of Gram-negative bacteria, such as E. coli or P. aeruginosa. The peptidoglycan structure of a bacterial cell wall is overall largely conserved with minor modifications (Schleifer & Kandler 1972). Bacterial species have interpeptide bridges composed of different amino acids or may even lack an interpeptide bridge. In peptidoglycan structures lacking an interpeptide bridge a Diaminopimelic acid (DAP) or meso-Diaminopimelic acid (mDAP; an amino acid, representing an epsilon-carboxy derivative of lysine being a typical component of peptidoglycan) (Diaminopimelic acid is residue replaces the amino acid L-Lys and directly cross-links to the terminal amino acid D-Ala of the opposite peptide chain. Thus, there are limited types of chemical bonds available that can be cleaved by muralytic enzymes (e.g. hydrolyzed by peptidoglycan hydrolases). The muralytic enzymes exhibit at least one enzyme domain having an enzymatic activity as listed above. In addition the muralytic enzymes contain in some cases at least one domain suitable for binding to the peptidoglycan and supporting the enzymatic activity of the muralytic enzyme. The binding domains are typically called cell-wall binding domains (CBD). Examples of muralytic enzymes are vertebrate lysozymes (such as hen egg white lysozyme and human lysozyme), endolysins (e.g. KZ144 endolysin or Lys394 endolysin), Virion-associated peptidoglycan hydrolases (VAPGH), bacteriocins (e.g. lysostaphin) and autolysins. Most preferably, the muralytic enzyme is an endolysin. Particularly preferred endolysin sequences are those set out above for the first to sixth aspect of the invention, e.g. SEQ ID NO:6, SEQ ID NO:28, SEQ ID NO:29, SEQ ID NO:30, SEQ ID NO:31, SEQ ID NO:32, SEQ ID NO:33, SEQ ID NO:34, SEQ ID NO:35, and SEQ ID NO:36, or a sequence having at least 80% sequence identity, preferably at least 85% sequence identity, more preferably at least 90% sequence identity, more preferably at least 95% sequence identity, more preferably at least 96% sequence identity, more preferably at least 97% sequence identity, more preferably at least 98% sequence identity, more preferably at least 99% sequence identity with SEQ ID NO:6, SEQ ID NO:28, SEQ ID NO:29, SEQ ID NO:30, SEQ ID NO:31, SEQ ID NO:32, SEQ ID NO:33, SEQ ID NO:34, SEQ ID NO:35, and/or SEQ ID NO:36, in particular to SEQ ID NO:28 and/or SEQ ID NO:30. In general: what has been set out above for an inventive polypeptide (according to the first to sixth aspect of the invention) applies (to the extent applicable) likewise to an inventive polypeptide according to the seventh aspect of the invention.

An inventive polypeptide comprises or may comprise a Gram negative endolysin. A polypeptide of the invention will therefore preferably be capable of degrading the peptidoglycan of at least on Gram-negative bacterium, usually the host species of the respective parental phage. Preferably, a polypeptide of the present invention will be capable of degrading the peptidoglycan of E. coli bacteria and/or P. aeruginosa bacteria. Most preferably, a polypeptide of the present invention degrades the peptidoglycan of E. coli strain RKI 06-08410 (obtained from Robert Koch-Institut, Berlin, Germany).

Peptidoglycan degrading activity on Gram-negative bacteria can be measured by assays well known in the art, e.g. by muralytic assays in which the outer membrane of Gram-negative bacteria is permeabilized or removed (e.g. with chloroform) to allow the putative enzyme access to the peptidoglycan layer. If the enzyme is active, degradation of the peptidoglycan layer will lead to a drop of turbidity, which can be measured photometrically (see for example Briers et al., J. Biochem. Biophys Methods 70: 531-533, (2007) or Schmelcher et al., Bacteriophage endolysins as novel antimicrobials. Schmelcher M, Donovan D M, Loessner M J. Future Microbiol. 2012 October; 7(10):1147-7) (both references incorporated herein by reference).

A polypeptide of the present invention will typically not only exhibit the activity of a peptidoglycan degrading enzyme, i.e. is capable of degrading Gram-negative bacterial peptidoglycan. Preferably, a polypeptide of the present invention will be capable of degrading the peptidoglycan of Gram-negative bacteria (e.g. E. coli bacteria and/or P. aeruginosa bacteria) in absence of EDTA (or any other auxiliary substance increasing the permeability of the outer membrane). More preferably, the inventive polypeptide exhibits a minimal inhibitory concentration (MIC) of 40 μg/ml or less in absence of other outer membrane permeabilizers), preferably of 30 μg/ml or less, even more preferably of 25 μg/ml or less even more preferably of 20 μg/ml or less, most preferably of 15 μg/ml or less. Most preferably, the polypeptide degrades the peptidoglycan of E. coli strain RKI 06-08410 with a minimal inhibitory concentration (MIC) of 40 μg/ml or less in absence of other outer membrane permeabilizers), preferably of 30 μg/ml or less, more preferably of 25 μg/ml or less, even more preferably of 20 μg/ml or less, most preferably of 15 μg/ml or less. A corresponding suitable test is set forth in Example 1. For P. aeruginosa, a suitable test is set forth in Example 2 and the respective test strain is preferably PAO1 (Pirnay et al., Environmental Microbiology, 2002, p. 898-911).

A polypeptide according to the present invention can be produced by standard means known in the art, e.g. by recombinant expression of nucleic acids encoding the respective polypeptide in appropriate host cells. If the inventive polypeptide comprises for example additionally amino acid sequence stretches or tags etc., such fusion proteins may be produced by linking the required individual nucleic acid sequences using standard cloning techniques as described e.g. by Sambrook et al. 2001, Molecular Cloning: A Laboratory Manual. Such a polypeptide may be produced likewise with methods known in the art, e.g., in recombinant DNA expression systems.

The present invention does also relate to nucleic acids encoding one or more inventive polypeptides of the present invention. The inventive nucleic acid may take all forms conceivable for a nucleic acid. In particular the nucleic acids according to the present invention may be RNA, DNA or hybrids thereof. They may be single-stranded or double-stranded. The may have the size of small transcripts or of entire genomes, such as a bacteriophage genome. As used herein, a nucleic acid encoding one or more inventive polypeptides of the present invention may be a nucleic acid reflecting the sense strand. Likewise, the antisense strand is also encompassed. The nucleic acid may encompass a heterologous promotor for expression of the inventive polypeptide.

In a further aspect the present invention relates to a vector comprising a nucleic acid according to the present invention. Such vector may for example be an expression vector allowing for expression of an inventive polypeptide. Said expression vector may be constitutive or inducible. The vector may also be a cloning vector comprising the nucleic acid sequence of the current invention for cloning purposes.

The present invention does also relate to a bacteriophage comprising an inventive nucleic acid, in particular comprising an inventive nucleic acid encoding a fusion protein according to the present invention.

The present invention does also relate to (isolated) host cells comprising a polypeptide, nucleic acid, vector, or bacteriophage according to the present invention. The host cells may be selected in particular from the group consisting of bacterial cells and yeast cells. Where appropriate, other suitable host cells may be immortalized cell lines, e.g. of mammalian (in particular human) origin.

In a further aspect the present invention relates to a composition comprising a polypeptide according to the present invention, a nucleic acid according to the present invention, a vector according to the present invention, a bacteriophage according to the present invention and/or a host cell according to the present invention. A composition according to the present invention may be a pharmaceutical composition comprising a pharmaceutical acceptable diluent, excipient or carrier. Particularly preferred are compositions comprising a polypeptide according to the present invention but are free of EDTA. Preferably, the composition of the invention is free of any other outer membrane permeabilizing substance.

In an even further aspect the composition according to the present invention is a cosmetic composition. Several bacterial species can cause irritations on environmentally exposed surfaces of the patient's body such as the skin. In order to prevent such irritations or in order to eliminate minor manifestations of said bacterial pathogens, special cosmetic preparations may be employed, which comprise sufficient amounts of the inventive polypeptide, nucleic acid, vector, host cell and/or composition in order to achieve a comedolytic effect. Preferably, the inventive polypeptide, nucleic acid, vector, bacteriophage, host cell or composition is used in this context without any other outer membrane permeabilizing substance.

In a further aspect the present invention relates to a kit comprising a polypeptide according to the present invention, a nucleic acid according to the present invention, a vector according to the present invention, a bacteriophage according to the present invention and/or a host cell according to the present invention, and at least one further antimicrobial agent, such as a further polypeptide according to the present invention, an antibiotic or an antimicrobial peptide.

In a further aspect the present invention relates to a polypeptide according to the present invention, a nucleic acid according to the present invention, a vector according to the present invention, a bacteriophage according to the present invention, a host cell according to the present invention, and/or a composition according to the present invention for use in a method of treatment of the human or animal body by surgery or therapy or in diagnostic methods practiced on the human or animal body. In such scenarios the antibacterial activity of polypeptide of the present invention can be exploited.

Such method typically comprises administering to a subject an effective amount of an inventive polypeptide, nucleic acid, vector, bacteriophage, host cell or a composition. Preferably the polypeptide, nucleic acid, vector, bacteriophage, host cell or a composition is administered without addition of further outer membrane permeabilizing substances such as EDTA. The subject may for example be a human or an animal, with human subjects being more preferred. In particular, the inventive polypeptide, the inventive nucleic acid, the inventive vector, the inventive bacteriophage, the inventive host cell, and/or the inventive composition may be used in methods for the treatment or prevention of bacterial infections, such Gram-negative bacterial infections. Without being limited thereto, the method of treatment may comprise the treatment and/or prevention of infections of the skin, of soft tissues, the respiratory system, the lung, the digestive tract, the eye, the ear, the teeth, the nasopharynx, the mouth, the bones, the vagina, of wounds of bacteraemia and/or endocarditis.

The dosage and route of administration used in a method of treatment (or prophylaxis) according to the present invention depends on the specific disease/site of infection to be treated. The route of administration may be for example oral, topical, nasopharyngeal, parenteral, intravenous, rectal or any other route of administration.

For application of an inventive polypeptide, nucleic acid, vector, bacteriophage, host cell or composition to a site of infection (or site endangered to be infected) a formulation may be used that protects the active compounds from environmental influences such as proteases, oxidation, immune response etc., until it reaches the site of infection. Therefore, the formulation may be capsule, dragee, pill, suppository, injectable solution or any other medical reasonable galenic formulation. Preferably, the galenic formulation may comprise suitable carriers, stabilizers, flavourings, buffers or other suitable reagents. For example, for topical application the formulation may be a lotion or plaster, for nasopharyngeal application the formulation may be saline solution to be applied via a spray to the nose. Preferably, the formulation does not comprise any other outer membrane permeabilizing substance (other than the inventive polypeptide).

Preferably, an inventive polypeptide, nucleic acid, vector, bacteriophage, host cell or composition is used in combination with other conventional antibacterial agents, such as antibiotics, lantibiotics, bacteriocins or endolysins, etc. The administration of the conventional antibacterial agent can occur prior to, concurrent with or subsequent to administration of the inventive polypeptide (e.g. fusion protein), nucleic acid, vector, bacteriophage, host cell or composition.

In a further aspect the present invention relates to the inventive polypeptide, nucleic acid, vector, bacteriophage, host cell or composition for use as diagnostic means in medical diagnostics, food diagnostics, feed diagnostics, or environmental diagnostics, in particular as a diagnostic means for the diagnostic of bacterial infection, in particular those caused by Gram-negative bacteria. In this respect the inventive polypeptide, nucleic acid, vector, host cell or composition may be used as a tool to specifically degrade the peptidoglycan of Gram-negative pathogenic bacteria. Specific cell degradation is needed as an initial step for subsequent specific detection of bacteria using nucleic acid based methods like PCR, nucleic acid hybridization or NASBA (Nucleic Acid Sequence Based Amplification), immunological methods like IMS, immunofluorescence or ELISA techniques, or other methods relying on the cellular content of the bacterial cells like enzymatic assays using proteins specific for distinct bacterial groups or species (e.g. β-galactosidase for enterobacteria, coagulase for coagulase positive strains). Preferably, the inventive polypeptide, nucleic acid, vector, bacteriophage, host cell or composition is used in this context without any other outer membrane permeabilizing substance.

In a further aspect the present invention relates to the use of the inventive polypeptide, the inventive nucleic acid, the inventive vector, the inventive bacteriophage, the inventive host cell, and/or the inventive composition, as an antimicrobial in food, in feed, or in cosmetics, or as a (e.g., non-therapeutic) disinfecting agent. An inventive polypeptide can be used for the treatment or prevention of Gram-negative bacterial contamination of foodstuff, of food processing equipment, of food processing plants, of (inanimate) surfaces coming into contact with foodstuff (such as shelves and food deposit areas), of feedstuff, of feed processing equipment, of feed processing plants, of (inanimate) surfaces coming into contact with feedstuff (such as shelves and feed deposit areas), of medical devices, or of (inanimate) surfaces in hospitals, doctor's offices and other medical facilities. Preferably, the inventive polypeptide, nucleic acid, vector, bacteriophage, host cell or composition is used in this context without any other outer membrane permeabilizing substance.

### FIGURE

In the following a brief description of the appended figures will be given. The figures are intended to illustrate an aspect of the present invention in more detail. However, it is not intended to limit the subject matter of the invention to such subject-matter only.

FIG. 1: illustrates the joint motif (boxed) of SEQ ID NO:2 found in preferred endolysin components of the polypeptides of the invention, namely endolysins of Citrobacter koseri phage CkP1 (SEQ ID NO:21), Enterobacteria phage CC31 (SEQ ID NO:22), Serratia phage CHI14 (SEQ ID NO:21), Aeromonas phage Ah1 (SEQ ID NO:24), Serratia phage PS2 (SEQ ID NO:25), and Aeromonas phage AS-szw (SEQ ID NO:26), giving rise to the consensus sequence of SEQ ID NO:7.

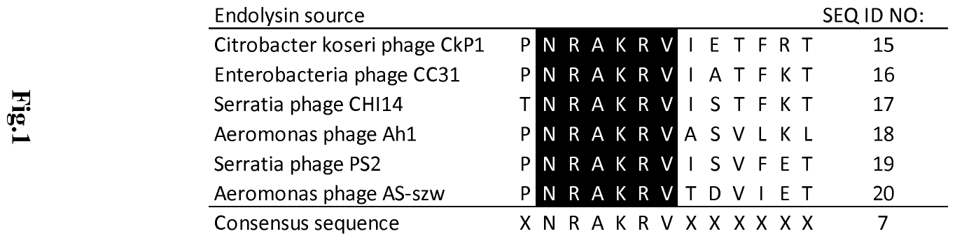

FIG. 2: illustrates positional requirements of preferred sequence motifs of selected peptide components of the inventive polypeptide. The table indicates for sequence motifs of 16 (white) to 20 (dark grey) amino acids in length positions at which no amino acid selected from the first group may be present (respective positions are labelled with “X”). At said positions (i.e. those labelled with “X”), only amino acids selected from the second, or as the case may be, from the third group may be present. More preferably, only amino acids selected from the second group are present at said positions. Amino acids selected from the first group of the sequence motif may only be present at positions which are not labelled with an “X”. However, at said non-labelled positions, amino acids of the second, or as the case may be, third group may also be present. Altogether 18 alternatives, each for a length of 16, 17, 18, 19 or 20 amino acids are provided. The table also clearly specifies the position where potentially a triplet amino acid of the first group may be present (three positions in a row without “X”). For alternative 1 this would be positions 8 to 10. As required for the preferred sequence motif of the peptide component polypeptide of the present invention, the amino acids at positions −5 (i.e. position #3), −4 (i.e. position #4), +6 (i.e. position #14), +7 (i.e. position #15), and +10 (i.e. position #18) relative to the first amino acid of the 3 amino acid block (i.e. position #8) are not to be selected from the first group. The relative positions 12, −11, −8, +13, and +14 cannot be found in the first alternative and are thus not taken into account.

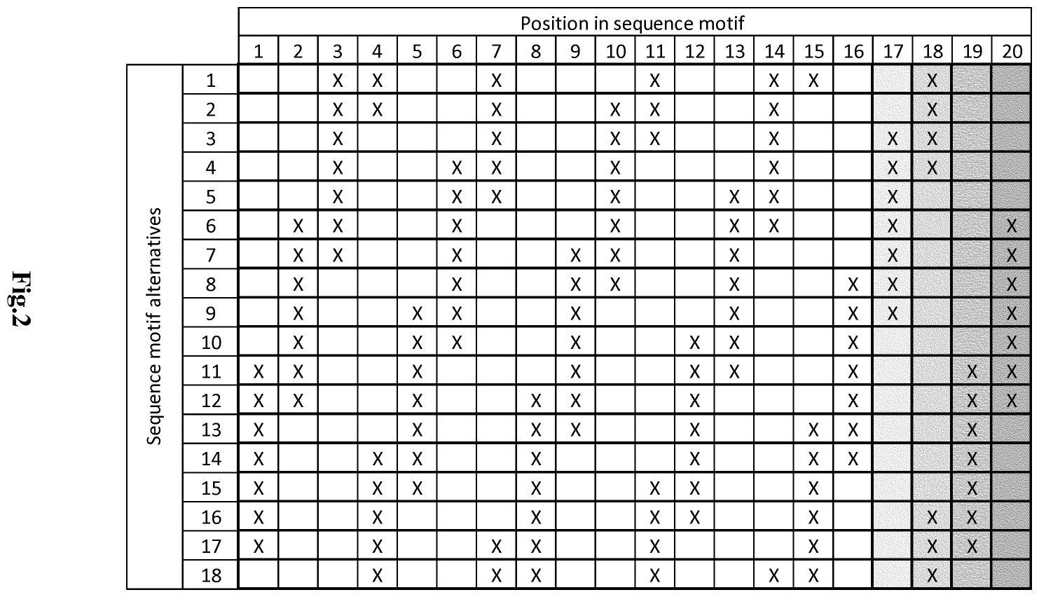

### EXAMPLES

In the following a specific example illustrating embodiments and aspects of the invention is presented. However, the present invention shall not to be limited in scope by the specific embodiments described herein. Indeed, various modifications of the invention in addition to those described herein will become readily apparent to those skilled in the art from the foregoing description and the example below. All such modifications fall within the scope of the appended claims.

### Example 1: Minimal Inhibitory Concentration of Several Antibacterial Polypeptides against E. coli in Presence and Absence of EDTA

The antibacterial activity of the following fusion proteins on E. coli in presence and absence of EDTA was assessed:

SEQ ID NO:154, a fusion of Cecropin A. (A aegyptii) peptide (SEQ ID NO: 69) with the endolysin of Vibrio phage VvAW1 (YP_007518361.1)

SEQ ID NO:155, a fusion of Cecropin A. (A aegyptii) peptide with a mutated cell wall binding domain of the modular KZ144 endolysin and Lys68 endolysin

SEQ ID NO:156, a fusion of a modified peptide (SEQ ID NO:131) complying with the preferred sequence motif of the peptide component and an endolysin of Pseudomonas phage vB_PsyM_KIL1 (see YP_009276009.1)

SEQ ID NO:140, a fusion of SMAP-29 peptide (SEQ ID NO:63) and the endolysin of Citrobacter koseri phage CkP1, with the additional technical modification of a C54S mutation to reduce aggregation (SEQ ID NO:28), and

SEQ ID NO:141, a fusion of a peptide comprising the preferred sequence motif for the peptide component (SEQ ID NO:132) and the endolysin of Enterobacteria phage CC31, with the additional technical modification of a C54S mutation to reduce aggregation (SEQ ID NO:30)

SEQ ID NO:142, a fusion of the peptide according to SEQ ID NO: 133 and the endolysin of Citrobacter koseri phage CkP1, with the additional technical modification of a C54S mutation to reduce aggregation (SEQ ID NO:28),

SEQ ID NO:143, a fusion of a peptide comprising a preferred sequence according to SEQ ID NO:134 and the endolysin of Enterobacteria phage CC31, with again the additional technical modification of a C54S mutation to reduce aggregation (SEQ ID NO:30),

SEQ ID NO:145, a fusion of a peptide comprising a preferred sequence according to SEQ ID NO:107 and the endolysin of Serratia phage CHI14, with again the additional technical modification of a C54S mutation to reduce aggregation (SEQ ID NO:32),

SEQ ID NO:146, a fusion of a peptide comprising a preferred sequence according to SEQ ID NO:SEQ ID NO:132 and the endolysin of Aeromonas phage Ah1, with again an additional technical modification of cysteine residue, here at position 122, to reduce aggregation (SEQ ID NO:34),

SEQ ID NO:147, a fusion of a peptide comprising a preferred sequence according to SEQ ID NO: 107 and the endolysin of Aeromonas phage Ah1, with again a additional technical modification of cysteine residue, here at position 122 of the wildtype endolysin sequence, to reduce aggregation (SEQ ID NO:34),

SEQ ID NO:148, a fusion of a peptide comprising a preferred sequence according to SEQ ID NO:132 and the endolysin of Serratia phage PS2 (SEQ ID NO:25),

SEQ ID NO:149, a fusion of a peptide comprising a preferred sequence according to SEQ ID NO:96 and the endolysin of Serratia phage PS2 (SEQ ID NO:25),

SEQ ID NO:150, a fusion of a peptide comprising a preferred sequence according to SEQ ID NO:135 and the endolysin of Citrobacter koseri phage CkP1, with the additional technical modification of a C54S mutation to reduce aggregation (SEQ ID NO:28), and

SEQ ID NO:152, a fusion of a peptide comprising a preferred sequence according to SEQ ID NO: SEQ ID NO:132 and the endolysin of Aeromonas phage AS-szw (SEQ ID NO:36),

The endolysin components of the polypeptide of SEQ ID NO:140 and the polypeptide of SEQ ID NO:141, (see SEQ ID NO:28 and SEQ ID NO:30) share a significant level of sequence identity (80%). Noteworthy, both endolysins share a relatively good conserved helical motif in the C-terminus (aa145-157), which is not present in any of the other fusion proteins tested.

E. coli bacteria (E. coli strain RKI 06-08410; obtained from Robert Koch-Institut, Berlin, Germany) were grown in (Luria-Bertani) medium and diluted 1:10 in Mueller-Hinton medium. At an optical density OD₆₀₀ of about 0.6 bacteria were diluted in the same medium 1:10 followed by a 1:500 dilution. Protein buffer (20 mM HEPES, 150 mM NaCl, pH 7.4) and proteins were pipetted into a 96 well plate using different concentrations of proteins in an end volume of 20 μl with or without a final concentration of 500 μM EDTA. 180 μl of bacterial cell suspension or medium (Mueller-Hinton) as control were given to the 96 well plate and mixed. The plate was incubated for 18-22 hours at 37° C. and the bacterial growth was determined measuring the OD600 values of the wells. The minimal inhibitory concentration (MIC), which is the protein concentration in the well which showed the same OD600 value as the no-bacteria control, was determined.

The results in form of minimal inhibitory concentration (MIC in μg/ml) are shown in table 4 below.

TABLE 4
Antibacterial activity in presence and absence of EDTA

| | MIC (μg/ml) | MIC (μg/ml) |
|---|---|---|
| SEQ ID NO | with EDTA | w/o EDTA |
| SEQ ID NO: 154 | ≤5 | 25 |
| SEQ ID NO: 155 | ≤5 | >50 |
| SEQ ID NO: 156 | ≤5 | >50 |
| SEQ ID NO: 140 | ≤5 | 10 |
| SEQ ID NO: 141 | ≤5 | 10 |
| SEQ ID NO: 142 | ≤5 | 15 |
| SEQ ID NO: 143 | ≤5 | 10 |
| SEQ ID NO: 145 | ≤5 | 7.5 |
| SEQ ID NO: 146 | 7.5 | 7.5 |
| SEQ ID NO: 147 | ≤5 | 15 |
| SEQ ID NO: 148 | 2.5 | 10 |
| SEQ ID NO: 149 | ≤5 | 5 |
| SEQ ID NO: 150 | ≤3.3 | 16.7 |
| SEQ ID NO: 152 | ≤5 | 12.5 |

“≤” (e.g. ≤5, ≤3.3 or the like) means, that antibacterial activity was observed already at the first concentration tested (e.g., 5 μg/ml and 3.3 μg/ml, respectively). The MIC is thus at least the first tested concentration (e.g. 5 μg/ml and 3.3 μg/ml, respectively) and possibly lower. >50 means, that no antibacterial activity could be observed up to a concentration of 50 μg/ml.

All polypeptides tested showed good antibacterial activity against E. coli in presence of the outer membrane permeabilizer EDTA. However, in absence of EDTA, the antibacterial activity for three conventional fusion proteins dropped significantly. In contrast, the polypeptides according to the present invention retained a significant level of antimicrobial activity even in absence of EDTA.

### Example 2: Minimal Inhibitory Concentration of Several Antibacterial Polypeptides against P. aeruginosa in Presence and Absence of EDTA

The antibacterial activity on P. aeruginosa bacteria in presence and absence of EDTA was also assessed. The following polypeptides were used:

SEQ ID NO:154 , a fusion of Cecropin A. (A aegyptii) peptide (SEQ ID NO: 69) with the endolysin of Vibrio phage VvAW1 (YP_007518361.1)

SEQ ID NO:155, a fusion of Cecropin A. (A aegyptii) peptide with a mutated cell wall binding domain of the modular KZ144 endolysin and Lys68 endolysin

SEQ ID NO:156, a fusion of a modified peptide (SEQ ID NO:131) complying with the preferred sequence motif of the peptide component and an endolysin of Pseudomonas phage vB_PsyM_KIL1 (see YP_009276009.1)

SEQ ID NO:140, a fusion of SMAP-29 peptide (SEQ ID NO:63) and the endolysin of Citrobacter koseri phage CkP1, with the additional technical modification of a C54S mutation to reduce aggregation (SEQ ID NO:28), and

SEQ ID NO:141, a fusion of a peptide comprising the preferred sequence motif for the peptide component (SEQ ID NO:132) and the endolysin of Enterobacteria phage CC31, with the additional technical modification of a C54S mutation to reduce aggregation (SEQ ID NO:30)

SEQ ID NO:144, a fusion of a peptide comprising a preferred sequence according to SEQ ID NO: SEQ ID NO:132 and the endolysin of Serratia phage CHI14, with again the additional technical modification of a C54S mutation to reduce aggregation (SEQ ID NO:32),

SEQ ID NO:145, a fusion of a peptide comprising a preferred sequence according to SEQ ID NO:107 and the endolysin of Serratia phage CHI14, with again the additional technical modification of a C54S mutation to reduce aggregation (SEQ ID NO:32),

SEQ ID NO:146, a fusion of a peptide comprising a preferred sequence according to SEQ ID NO:SEQ ID NO:132 and the endolysin of Aeromonas phage Ah1, with again a additional technical modification of cysteine residue, here at position 122, to reduce aggregation (SEQ ID NO:34),

SEQ ID NO:147, a fusion of a peptide comprising a preferred sequence according to SEQ ID NO: 107 and the endolysin of Aeromonas phage Ah1, with again a additional technical modification of cysteine residue, here at position 122 of the wildtype endolysin sequence, to reduce aggregation (SEQ ID NO:34),

SEQ ID NO:148, a fusion of a peptide comprising a preferred sequence according to SEQ ID NO:132 and the endolysin of Serratia phage PS2 (SEQ ID NO:25),

SEQ ID NO:149, a fusion of a peptide comprising a preferred sequence according to SEQ ID NO:96 and the endolysin of Serratia phage PS2 (SEQ ID NO:25),

SEQ ID NO:150, a fusion of a peptide comprising a preferred sequence according to SEQ ID NO:135 and the endolysin of Citrobacter koseri phage CkP1, with the additional technical modification of a C54S mutation to reduce aggregation (SEQ ID NO:28),

SEQ ID NO:151, a fusion of a peptide comprising a preferred sequence according to SEQ ID NO:136 and the endolysin of Citrobacter koseri phage CkP1, with the additional technical modification of a C54S mutation to reduce aggregation (SEQ ID NO:28), and

SEQ ID NO:152, a fusion of a peptide comprising a preferred sequence according to SEQ ID NO: SEQ ID NO:132 and the endolysin of Aeromonas phage AS-szw (SEQ ID NO:36).

Bacteria (P. aeruginosa PAO1) were grown in (Luria-Bertani) medium and diluted 1:10 in Mueller-Hinton medium. At an optical density OD₆₀₀ of about 0.6 bacteria were diluted in the same medium 1:10 followed by a 1:500 dilution. Protein buffer (20 mM HEPES, 150 mM NaCl, pH 7.4) and proteins were pipetted into a 96 well plate using different concentrations of proteins in an end volume of 20 μl with or without a final concentration of 500 μM EDTA. 180 μl of bacterial cell suspension or medium (Mueller-Hinton) as control were given to the 96 well plate and mixed. The plate was incubated for 18-22 hours at 37° C. and the bacterial growth was determined measuring the OD600 values of the wells. The MIC which is the protein concentration in the well which showed the same OD600 value as the no-bacteria control was determined.

The results in form of minimal inhibitory concentration (MIC in μg/ml) are shown in table 5 below.

TABLE 5
Antibacterial activity in presence and absence of EDTA

| | MIC (μg/ml) | MIC (μg/ml) |
|---|---|---|
| SEQ ID NO | with EDTA | w/o EDTA |
| SEQ ID NO: 154 | ≤5 | >50 |
| SEQ ID NO: 155 | ≤5 | >50 |
| SEQ ID NO: 156 | 10 | >50 |
| SEQ ID NO: 140 | ≤5 | 5 |
| SEQ ID NO: 141 | ≤5 | 10 |
| SEQ ID NO: 144 | ≤4.5 | 6.8 |
| SEQ ID NO: 145 | ≤5 | 7.5 |
| SEQ ID NO: 146 | ≤5 | 7.5 |
| SEQ ID NO: 147 | ≤5 | 7.5 |
| SEQ ID NO: 148 | ≤1.5 | 10 |
| SEQ ID NO: 149 | ≤5 | 10 |
| SEQ ID NO: 150 | ≤3.3 | 8.3 |
| SEQ ID NO: 151 | ≤5 | 15 |
| SEQ ID NO: 152 | ≤5 | 5 |

“≤” (e.g. ≤5, ≤1.5 or the like) means, that antibacterial activity was observed already at the first concentration tested (e.g. 5 μg/ml and 1.5 μg/ml, respectively). The MIC is thus at least the first tested concentration (e.g. 5 μg/ml and 1.5 μg/ml, respectively) and possibly lower. >50 means, that no antibacterial activity could be observed up to a concentration of 50 μg/ml.

All polypeptides tested showed good antibacterial activity against P. aeruginosa in presence of the outer membrane permeabilizer EDTA. However, in absence of EDTA, the antibacterial activity for three conventional fusion proteins dropped significantly. In contrast, the polypeptides according to the present invention retained a significant level of antimicrobial activity even in absence of EDTA.

### Example 3: Preferred Embodiments of the First, Third and Fifth Aspect of the Invention

1. Polypeptide comprising a Gram negative endolysin and a peptide selected from the group consisting of an antimicrobial peptide, an amphipathic peptide, a cationic peptide, a sushi peptide or a defensin,

wherein the endolysin in turn is an endolysin comprising a sequence according to SEQ ID NO:1,

with the provisos that:

a) the polypeptide does neither comprise the sequence according to SEQ ID NO:3 nor according to SEQ ID NO:4 nor according to SEQ ID NO:5,

b) the endolysin is neither Aeh1p339 of Aeromonas phage Aeh1 nor EpJS98_gp116 of Enterobacteria phage JS98,

c) the peptide is selected from the group consisting of an antimicrobial peptide, an amphipathic peptide, a sushi peptide or a defensin, if the polypeptide comprises the sequence of SEQ ID NO:6,

d) the polypeptide does not comprise a cell wall binding domain of i) a modular Gram-negative endolysin or ii) a bacteriophage tail/baseplate protein, if the endolysin has a sequence selected from the group consisting of:

| Host | Phage name | Protein ID |
|---|---|---|
| Aeromonas | Aeromonas phage PX29 | ADQ53036.1 |
| Aeromonas | Aeromonas phage phiAS4 | YP_003969055.1 |
| Aeromonas | Aeromonas phage 44RR2.8t | NP_932578.1 |
| Aeromonas | Aeromonas phage 25 | YP_656449.1 |
| Aeromonas | Aeromonas phage 31 | YP_238949.1 |
| Aeromonas | Aeromonas phage 65 | YP_004300997.1 |
| Aeromonas | Aeromonas phage phiAS5 | YP_003969406.1 |
| Escherichia | Escherichia phage wV7 | AEM00790.1 |
| Escherichia | Enterobacteria phage vB_EcoM-VR7 | YP_004063811.1 |
| Escherichia | Enterobacteria phage Bp7 | AEN93735.1 |
| Escherichia | Enterobacteria phage AR1 | BAI83135.1 |
| Escherichia | Enterobacteria phage JS10 | YP_002922463.1 |
| Escherichia | Enterobacteria phage IME08 | YP_003734260.1 |
| Escherichia | Enterobacteria phage CC31 | YP_004009990.1 |
| Escherichia | Enterobacteria phage RB69 | NP_861818.1 |
| Escherichia | Enterobacteria phage RB14 | YP_002854463.1 |
| Escherichia | Enterobacteria phage RB32 | ABI94948.1 |
| Escherichia | Enterobacteria phage RB51 | YP_002854084.1 |
| Shigella | Shigella phage Shfl2 | YP_004415022.1 |

and corresponding sequences merely lacking in addition the N-terminal methionine,

e) the polypeptide does not comprise a cell wall binding domain of i) a modular Gram-negative endolysin or ii) a bacteriophage tail/baseplate protein, if the endolysin has a sequence according to amino acids 2-164 of:

| Host | Phage name | Protein ID |
|---|---|---|
| Escherichia | Enterobacteria phage T4 | NP_049736.1 |

f) the polypeptide does not comprise a cell wall binding domain of i) a modular Gram-negative endolysin or ii) a bacteriophage tail/baseplate protein, if the endolysin has a sequence according to amino acids 2-165 of:

| Host | Phage name | Protein ID |
|---|---|---|
| Aeromonas | Aeromonas phage Aeh1 | NP_944217.1 |

g) the polypeptide does not comprise a cell wall binding domain of i) a modular Gram-negative endolysin or ii) a bacteriophage tail/baseplate protein, if the endolysin has a sequence according to amino acids 2-161 of:

| Host | Phage name | Protein ID |
|---|---|---|
| Escherichia | Enterobacteria phage JS98 | YP_001595245.1 |

2. The polypeptide according to item 1, wherein the endolysin is selected from the group consisting of SEQ ID NO:21, SEQ ID NO:22, SEQ ID NO:28, SEQ ID NO:30 and sequences having at least 80% sequence identity with SEQ ID NO:21, SEQ ID NO:22,SEQ ID NO:28 and/or SEQ ID NO:30.
3. The polypeptide according to item 1, wherein SEQ ID NO:1 is further defined as having a sequence selected from the group consisting of SEQ ID NO:8, SEQ ID NO:9, SEQ ID NO:10 and 11.
4. The polypeptide according to any one of the preceding items, wherein the peptide is an antimicrobial peptide or an amphipathic peptide.
5. The polypeptide according to any one of the preceding items, wherein the peptide comprises a sequence motif which:

i) is 16, 17, 18, 19 or 20 amino acids in length;

ii) comprises at least 40% and at most 60% amino acids selected from a first group of amino acids consisting of lysine, arginine and histidine, wherein each amino acid is selected independently from said first group, wherein each amino acid selected from this first group is arranged in said sequence motif either alone, pairwise together with a further amino acid selected from the first group, or in a block with 2 further amino acids selected from the first group, but does not occur in a block with 3 or more amino acids selected from the first group, wherein at least 2 pairs of amino acids selected from the first group are present in said sequence motif, and wherein at most one block with 3 of the amino acids selected from the first group in a row is present in said sequence motif, with the additional proviso, that if such block with 3 amino acids of the first group is present in said sequence motif, then the amino acids at positions −12, −11, −8, −5, −4, +6, +7, +10, +13, and +14 relative to the first amino acid of the 3 amino acid block are, provided the respective position may be found in said sequence motif, not selected from said first group,

iii) comprises at least 40% and at most 60% amino acids selected from a second group of amino acids consisting of alanine, glycine, isoleucine, leucine, phenylalanine, serine, threonine, tryptophan, tyrosine and valine, wherein each amino acid is selected independently from said second group, wherein preferably at least three different amino acids are selected from this second group, if the sum of amino acids of selected from the first group and selected from the second group yield 100% of the sequence motif;

iv) wherein the remaining amino acids of said sequence motif, if any are present in the motif, are selected from a third group consisting of asparagine, aspartic acid, glutamine, glutamic acid, methionine, or cysteine, wherein each of said amino acids is selected independently from said third group.

6. The polypeptide according to item 5, wherein peptide comprises the sequence according to SEQ ID NO:63 or according to SEQ ID NO:132.
7. The polypeptide according to any one of the preceding items, wherein the polypeptide comprises the amino acid sequence of SEQ ID NO:140 or of SEQ ID NO:141 or a sequence sharing at least 80% sequence identity with SEQ ID NO:140 and/or SEQ ID NO:141.
8. The polypeptide according to any one of the preceding items, wherein the polypeptide degrades peptidoglycan of at least one Gram-negative bacterial species, in particular wherein the polypeptide degrades the peptidoglycan of E. coli bacteria and/or P. aeruginosa bacteria.
9. The polypeptide according to item 8, wherein the polypeptide degrades the peptidoglycan of at least one Gram-negative bacterial species in absence of other outer membrane permeabilizing substances, in particular wherein the polypeptide degrades the peptidoglycan of E. coli bacteria and/or P. aeruginosa bacteria in absence of outer membrane permeabilizing substances.
10. The polypeptide according to item 8, wherein the polypeptide exhibits in absence of outer membrane permeabilizing substances a minimal inhibitory concentration (MIC) of 20 μg/ml or less for E. coli strain RKI 06-08410.
11. Nucleic acid encoding a polypeptide according to any one of items 1 to 10.
12. Vector comprising a nucleic acid according to item 11.
13. Host cell comprising a polypeptide according to any one of items 1 to 10, a nucleic acid according to item 11, and/or a vector according to item 12.
14. The polypeptide according to any one of items 1 to 10 for use in a method for treatment of the human or animal body by surgery or therapy or for use in diagnostic methods practiced on the human or animal body, wherein the polypeptide is administered without addition of further outer membrane permeabilizing substances.
15. Use of polypeptide according to any one of items 1 to 10 as non-therapeutic disinfectant, wherein the polypeptide is administered without addition of further outer membrane permeabilizing substances.

1. A polypeptide comprising a Gram negative endolysin and a peptide selected from the group consisting of an antimicrobial peptide, an amphipathic peptide, a cationic peptide, a sushi peptide or a defensin,
wherein the endolysin in turn is an endolysin comprising a sequence according to SEQ ID NO:2,
with the provisos that:
a) the polypeptide does neither comprise the sequence according to SEQ ID NO:3 nor according to SEQ ID NO:4 nor according to SEQ ID NO:5,
b) the endolysin is not EpJS98_gp116 of Enterobacteria phage J598,
c) the peptide is selected from the group consisting of an antimicrobial peptide, an amphipathic peptide, a sushi peptide or a defensin, if the polypeptide comprises the sequence of SEQ ID NO:6,
d) the polypeptide does not comprise a cell wall binding domain of i) a modular Gram-negative endolysin or ii) a bacteriophage tail/baseplate protein, if the endolysin has a sequence selected from the group consisting of:

| Host | Phage name | Protein ID |
|---|---|---|
| Aeromonas | Aeromonas phage 65 | YP_004300997.1 |
| Escherichia | Escherichia phage wV7 | AEM00790.1 |
| Escherichia | Enterobacteria phage vB_EcoM-VR7 | YP_004063811.1 |
| Escherichia | Enterobacteria phage Bp7 | AEN93735.1 |
| Escherichia | Enterobacteria phage AR1 | BAI83135.1 |
| Escherichia | Enterobacteria phage JS10 | YP_002922463.1 |
| Escherichia | Enterobacteria phage IME08 | YP_003734260.1 |
| Escherichia | Enterobacteria phage CC31 | YP_004009990.1 |
| Escherichia | Enterobacteria phage RB69 | NP_861818.1 |
| Escherichia | Enterobacteria phage RB14 | YP_002854463.1 |
| Escherichia | Enterobacteria phage RB32 | ABI94948.1 |
| Escherichia | Enterobacteria phage RB51 | YP_002854084.1 |
| Shigella | Shigella phage Shfl2 | YP_004415022.1 |

and corresponding sequences merely lacking in addition the N-terminal methionine,
e) the polypeptide does not comprise a cell wall binding domain of i) a modular Gram-negative endolysin or ii) a bacteriophage tail/baseplate protein, if the endolysin has a sequence according to amino acids 2-164 of:

| Host | Phage name | Protein ID |
|---|---|---|
| Escherichia | Enterobacteria phage T4 | NP_049736.1 |

f) the polypeptide does not comprise a cell wall binding domain of i) a modular Gram-negative endolysin or ii) a bacteriophage tail/baseplate protein, if the endolysin has a sequence according to amino acids 2-161 of:

| Host | Phage name | Protein ID |
|---|---|---|
| Escherichia | Enterobacteria phage JS98 | YP_001595245.1 |

2. The polypeptide according to claim 1, wherein the endolysin is an endolysin comprising a sequence according to SEQ ID NO:7.

3. The polypeptide according to claim 1, wherein the endolysin is selected from the group consisting of SEQ ID NO:6, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:.

4. The polypeptide according to claim 2, wherein the endolysin comprises a sequence selected from the group consisting of SEQ ID NO:8, SEQ ID NO:9, SEQ ID NO:10 and SEQ ID NO:11.

5. The polypeptide according to claim 1, wherein the peptide is an antimicrobial peptide or an amphipathic peptide.

6. The polypeptide according to claim 1, wherein the peptide comprises a sequence selected from the group consisting of KRK and SEQ ID NO: 37-136, in particular wherein the peptide comprises a sequence according to SEQ ID NO:63 or according to SEQ ID NO:132.

7. The polypeptide according to claim 1, wherein the polypeptide comprises the amino acid sequence of SEQ ID NO:140 or of SEQ ID NO:141.

8. A polypeptide comprising the sequence of a peptide selected from the group consisting of SEQ ID NO: 107, SEQ ID NO:133, SEQ ID NO:134, SEQ ID NO:135 and SEQ ID NO: 136 and the sequence of a muralytic enzyme.

9. The polypeptide according to claim 1, wherein the polypeptide degrades peptidoglycan of at least one Gram-negative bacterial species, in particular wherein the polypeptide degrades the peptidoglycan of E. coli bacteria and/or P. aeruginosa bacteria.

10. The polypeptide according to claim 9, wherein the polypeptide degrades the peptidoglycan of at least one Gram-negative bacterial species in absence of other outer membrane permeabilizing substances, in particular wherein the polypeptide degrades the peptidoglycan of E. coli bacteria and/or P. aeruginosa bacteria in absence of outer membrane permeabilizing substances.

11. The polypeptide according to claim 9, wherein the polypeptide exhibits in absence of outer membrane permeabilizing substances a minimal inhibitory concentration (MIC) of 20 μg/ml or less for E. coli strain RKI 06-08410.

12. A nucleic acid encoding a polypeptide according to claim 1.

13. A vector comprising a nucleic acid according to claim 12.

14. A host cell comprising a nucleic acid according to claim 12.

15. A method for treatment of a human or animal by surgery or therapy or a diagnostic method comprising administering to the human or animal the polypeptide of claim 1, wherein the polypeptide is administered without addition of further outer membrane permeabilizing substances.

16. A method of disinfecting a surface comprising contacting said surface with the polypeptide of claim 1, wherein the polypeptide is administered without addition of further outer membrane permeabilizing substances.

17. The polypeptide according to claim 8, wherein the polypeptide degrades peptidoglycan of at least one Gram-negative bacterial species, in particular wherein the polypeptide degrades the peptidoglycan of E. coli bacteria and/or P. aeruginosa bacteria.

18. A nucleic acid encoding a polypeptide according to claim 8.

19. A vector comprising a nucleic acid according to claim 18.


SEQUENCE LISTING (156 sequences)

SEQ ID NO: 1 - protein, 13 aa, Artificial sequence
  Consensus sequence
  MISC_FEATURE (1)..(1): Xaa can be Pro or Thr
  MISC_FEATURE (5)..(5): Xaa can be Lys, Met, Asn or Gln
  MISC_FEATURE (8)..(8): Xaa can be Ala, Ile or Thr
  MISC_FEATURE (9)..(9): Xaa can be Ala, Asp, Glu, Lys, Gln, Ser or Thr
  MISC_FEATURE (10)..(10): Xaa can be Val or Thr
  MISC_FEATURE (11)..(11): Xaa can be Phe, Ile, Val or Leu
  MISC_FEATURE (12)..(12): Xaa can be Glu, Lys, Leu or Arg
  MISC_FEATURE (13)..(13): Xaa can be  Leu or Thr
XNRAXRVXXXXXX

SEQ ID NO: 2 - protein, 6 aa, Enterobacteria phage CC31
NRAKRV

SEQ ID NO: 3 - protein, 163 aa, unknown
  Enterobacteria phage T4 endolysin
MNIFEMLRIDEGLRLKIYKDTEGYYTIGIGHLLTKSPSLNAAKSELDKAIGRNCNGVITKDEAEKLFNQDVDAAVRGILRNAKLKPVYDSLDAVRRCALINMVFQMGETGVAGFTNSLRMLQQKRWDEAAVNLAKSRWYNQTPNRAKRVITTFRTGTWDAYKN

SEQ ID NO: 4 - protein, 176 aa, unknown
  smi02_KRK9
AMGSKRKKRKKRKGNIFEMLRIDEGLRLKIYKDTEGYYTIGIGHLLTKSPSLNAAKSELDKAIGRNCNGVITKDEAEKLFNQDVDAAVRGILRNAKLKPVYDSLDAVRRCALINMVFQMGETGVAGFTNSLRMLQQKRWDEAAVNLAKSRWYNQTPNRAKRVITTFRTGTWDAYKN

SEQ ID NO: 5 - protein, 184 aa, Artificial sequence
  Recombninant endolysin derived from Citrobacter Koseri phage CkP1 including N-terminal HisTag
MGSSHHHHHHSSGLVPRGSHMNIFKMLRIDEGYDSKIYKDTEGFWTIGIGHLLTRDPSLEVAKRELDKLVGRKCNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAYK

SEQ ID NO: 6 - protein, 162 aa, Unknown
  Catalytic domain of endolysin derived from Citrobacter Koseri phage CkP1 w/o N-terminal methionine
NIFKMLRIDEGYDSKIYKDTEGFWTIGIGHLLTRDPSLEVAKRELDKLVGRKCNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAY

SEQ ID NO: 7 - protein, 13 aa, Artificial sequence
  Consensus sequence
  MISC_FEATURE (1)..(1): Xaa can be Pro or Thr
  MISC_FEATURE (8)..(8): Xaa can be Ala, Ile or Thr
  MISC_FEATURE (9)..(9): Xaa can be Ala, Asp, Glu, or Ser
  MISC_FEATURE (10)..(10): Xaa can be Val or Thr
  MISC_FEATURE (11)..(11): Xaa can be Phe, Ile or Leu
  MISC_FEATURE (12)..(12): Xaa can be Glu, Lys or Arg
  MISC_FEATURE (13)..(13): Xaa can be  Leu or Thr
XNRAKRVXXXXXX

SEQ ID NO: 8 - protein, 13 aa, Artificial sequence
  Consensus sequence
  MISC_FEATURE (1)..(1): Xaa can be Pro or Thr
  MISC_FEATURE (9)..(9): Xaa can be Ala or  Ser
  MISC_FEATURE (12)..(12): Xaa can be Lys or Arg
XNRAKRVIXTFXT

SEQ ID NO: 9 - protein, 13 aa, Artificial sequence
  Consensus sequence
  MISC_FEATURE (9)..(9): Xaa can be Ala or Ser
  MISC_FEATURE (12)..(12): Xaa can be Lys or Arg
PNRAKRVIXTFXT

SEQ ID NO: 10 - protein, 13 aa, Artificial sequence
  Consensus sequence
  MISC_FEATURE (1)..(1): Xaa can be Pro or Thr
  MISC_FEATURE (9)..(9): Xaa can be Ala, Glu or Ser
  MISC_FEATURE (12)..(12): Xaa can be Lys or Arg
XNRAKRVIXTFXT

SEQ ID NO: 11 - protein, 13 aa, Artificial sequence
  Consensus sequence
  MISC_FEATURE (9)..(9): Xaa can be Ala or Glu
  MISC_FEATURE (12)..(12): Xaa can be Lys or Arg
PNRAKRVIXTFXT

SEQ ID NO: 12 - protein, 13 aa, Aeromonas phage Aeh1
PNRANRVASVLKL

SEQ ID NO: 13 - protein, 13 aa, Aeromonas phage phiAS4
PNRAMRVAKVVLT

SEQ ID NO: 14 - protein, 13 aa, Aeromonas phage 44RR2.8t
PNRAQRVAQVILT

SEQ ID NO: 15 - protein, 13 aa, Unknown
  Sequence motif  of endolysin derived from Citrobacter Koseri phage CkP1
PNRAKRVIETFRT

SEQ ID NO: 16 - protein, 13 aa, Enterobacteria phage CC31
PNRAKRVIATFKT

SEQ ID NO: 17 - protein, 13 aa, Serratia phage CHI14
TNRAKRVISTFKT

SEQ ID NO: 18 - protein, 13 aa, Aeromonas phage Ah1
PNRAKRVASVLKL

SEQ ID NO: 19 - protein, 13 aa, Serratia phage PS2
PNRAKRVISVFET

SEQ ID NO: 20 - protein, 13 aa, Aeromonas phage As-szw
PNRAKRVTDVIET

SEQ ID NO: 21 - protein, 164 aa, Unknown
  Endolysin derived from Citrobacter Koseri phage CkP1
MNIFKMLRIDEGYDSKIYKDTEGFWTIGIGHLLTRDPSLEVAKRELDKLVGRKCNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAYK

SEQ ID NO: 22 - protein, 164 aa, Enterobacteria phage CC31
MDIFGMLRIDEGYDSKIYKDTEGFWTIGIGHLLTRDPSLDVAKRELDKLVGRPCNGQITKAEAEAIFAKDVDKATRGILGNAVLKPVYDVLDGVRRAALINMVFQMGVAGVASFPASMRLLKSKQWEAAAKELANSKWYRQTPNRAKRVIATFKTGTWKAYENL

SEQ ID NO: 23 - protein, 161 aa, Serratia phage CHI14
MDIFGMLRIDEGYDSKIYKDTEGYWTIGIGHLLTKNPSLSVAKAELDKLVGRSCNGQITQDEAESIFAKDVEKAVKGIQGNSVLKPVYDSLDEIRRAALINMVFQMGVAGVAGFTNSMRMLKEKRWDEAAVNLAKSRWYNQTTNRAKRVISTFKTGTWGAY

SEQ ID NO: 24 - protein, 165 aa, Aeromonas phage Ah1
MLAQMLKQDEGYKETVYWDTEGYPTIGIGHLILKKRTKDMGEINRELSSHVGRVVKDGKITGEEVLALFERDLSVLKRSIMSLPNLADVYVSLDMVRQTAIENMVFQMGAVGVSKFPGMLRCLKAKDWDGAYRNALDSAWARQTPNRAKRVASVLKLGSYAPYGF

SEQ ID NO: 25 - protein, 162 aa, Serratia phage PS2
MTIFEMLAFDEGLKLTVYLDTEGFWTVGIGHLLTKNPSKAVAIAELDKLVGRSTGGTITKAEAERIFAQDVAKSEKGIQGNAVLGPVYAGLDATRKMALVNMTFQLGVAGAAGFTNSMKLLAAKQWKEAAINLAKSKWYNQTPNRAKRVISVFETGTLAAYK

SEQ ID NO: 26 - protein, 166 aa, phage As-szw
MLEKMLKFDEGSKLSVYWDTEGYPTIGIGHLIKRLRTKDMGEINRELSSHVGRVITDGKITQSEESQLFAKDLEVVRNSMKGYVDLWSTYVGLDEVRKTALENMVFQMGAKGVNGFPSMLRAMRSKNWVEAKKHGLASAWSRQTPNRAKRVTDVIETGTYKGYPFA

SEQ ID NO: 27 - protein, 162 aa, unknown
  Enterobacteria phage T4 endolysin w/o N-terminal methionine
NIFEMLRIDEGLRLKIYKDTEGYYTIGIGHLLTKSPSLNAAKSELDKAIGRNCNGVITKDEAEKLFNQDVDAAVRGILRNAKLKPVYDSLDAVRRCALINMVFQMGETGVAGFTNSLRMLQQKRWDEAAVNLAKSRWYNQTPNRAKRVITTFRTGTWDAYKN

SEQ ID NO: 28 - protein, 163 aa, Artificial sequence
  Endolysin derived from Citrobacter Koseri phage CkP1, w/o N-terminal methionine and C54S
NIFKMLRIDEGYDSKIYKDTEGFWTIGIGHLLTRDPSLEVAKRELDKLVGRKSNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAYK

SEQ ID NO: 29 - protein, 163 aa, Enterobacteria phage CC31
DIFGMLRIDEGYDSKIYKDTEGFWTIGIGHLLTRDPSLDVAKRELDKLVGRPCNGQITKAEAEAIFAKDVDKATRGILGNAVLKPVYDVLDGVRRAALINMVFQMGVAGVASFPASMRLLKSKQWEAAAKELANSKWYRQTPNRAKRVIATFKTGTWKAYENL

SEQ ID NO: 30 - protein, 163 aa, Artificial sequence
  Endolysin derived from Enterobacteria phage CC31, w/o N-terminal methionine and C54S
DIFGMLRIDEGYDSKIYKDTEGFWTIGIGHLLTRDPSLDVAKRELDKLVGRPSNGQITKAEAEAIFAKDVDKATRGILGNAVLKPVYDVLDGVRRAALINMVFQMGVAGVASFPASMRLLKSKQWEAAAKELANSKWYRQTPNRAKRVIATFKTGTWKAYENL

SEQ ID NO: 31 - protein, 160 aa, Serratia phage CHI14
DIFGMLRIDEGYDSKIYKDTEGYWTIGIGHLLTKNPSLSVAKAELDKLVGRSCNGQITQDEAESIFAKDVEKAVKGIQGNSVLKPVYDSLDEIRRAALINMVFQMGVAGVAGFTNSMRMLKEKRWDEAAVNLAKSRWYNQTTNRAKRVISTFKTGTWGAY

SEQ ID NO: 32 - protein, 160 aa, Artificial sequence
  Endolysin derived from Serratia phage CHI14, w/o N-terminal methionine and C54S
DIFGMLRIDEGYDSKIYKDTEGYWTIGIGHLLTKNPSLSVAKAELDKLVGRSSNGQITQDEAESIFAKDVEKAVKGIQGNSVLKPVYDSLDEIRRAALINMVFQMGVAGVAGFTNSMRMLKEKRWDEAAVNLAKSRWYNQTTNRAKRVISTFKTGTWGAY

SEQ ID NO: 33 - protein, 164 aa, phage Ah1
LAQMLKQDEGYKETVYWDTEGYPTIGIGHLILKKRTKDMGEINRELSSHVGRVVKDGKITGEEVLALFERDLSVLKRSIMSLPNLADVYVSLDMVRQTAIENMVFQMGAVGVSKFPGMLRCLKAKDWDGAYRNALDSAWARQTPNRAKRVASVLKLGSYAPYGF

SEQ ID NO: 34 - protein, 164 aa, Artificial sequence
  Endolysin derived from Aeromonas phage Ah1, w/o N-terminal methionine and C122S
LAQMLKQDEGYKETVYWDTEGYPTIGIGHLILKKRTKDMGEINRELSSHVGRVVKDGKITGEEVLALFERDLSVLKRSIMSLPNLADVYVSLDMVRQTAIENMVFQMGAVGVSKFPGMLRSLKAKDWDGAYRNALDSAWARQTPNRAKRVASVLKLGSYAPYGF

SEQ ID NO: 35 - protein, 161 aa, Serratia phage PS2
TIFEMLAFDEGLKLTVYLDTEGFWTVGIGHLLTKNPSKAVAIAELDKLVGRSTGGTITKAEAERIFAQDVAKSEKGIQGNAVLGPVYAGLDATRKMALVNMTFQLGVAGAAGFTNSMKLLAAKQWKEAAINLAKSKWYNQTPNRAKRVISVFETGTLAAYK

SEQ ID NO: 36 - protein, 165 aa, Aeromonas phage As-szw
LEKMLKFDEGSKLSVYWDTEGYPTIGIGHLIKRLRTKDMGEINRELSSHVGRVITDGKITQSEESQLFAKDLEVVRNSMKGYVDLWSTYVGLDEVRKTALENMVFQMGAKGVNGFPSMLRAMRSKNWVEAKKHGLASAWSRQTPNRAKRVTDVIETGTYKGYPFA

SEQ ID NO: 37 - protein, 6 aa, artificial
  synthetic sequence
KRKKRK

SEQ ID NO: 38 - protein, 5 aa, artificial
  synethtic sequence
  misc_feature (3)..(3): Xaa can be any naturally occurring amino acid
KRXKR

SEQ ID NO: 39 - protein, 5 aa, artificial
  synthetic sequence
KRSKR

SEQ ID NO: 40 - protein, 5 aa, artificial
  synthetic sequence
KRGSG

SEQ ID NO: 41 - protein, 9 aa, artificial
  synthetic sequence
KRKKRKKRK

SEQ ID NO: 42 - protein, 9 aa, artificial
  synthetic sequence
RRRRRRRRR

SEQ ID NO: 43 - protein, 8 aa, artificial
  synthetic sequence
KKKKKKKK

SEQ ID NO: 44 - protein, 10 aa, artificial
  synthetic sequence
KRKKRKKRKK

SEQ ID NO: 45 - protein, 12 aa, artificial
  synthetic sequence
KRKKRKKRKKRK

SEQ ID NO: 46 - protein, 14 aa, artificial
  synthetic sequence
KRKKRKKRKKRKKR

SEQ ID NO: 47 - protein, 16 aa, artificial
  synthetic sequence
KKKKKKKKKKKKKKKK

SEQ ID NO: 48 - protein, 18 aa, artificial
  synthetic sequence
KRKKRKKRKKRKKRKKRK

SEQ ID NO: 49 - protein, 19 aa, artificial
  synthetic sequence
KRKKRKKRKKRKKRKKRKK

SEQ ID NO: 50 - protein, 19 aa, artificial
  synthetic sequence
RRRRRRRRRRRRRRRRRRR

SEQ ID NO: 51 - protein, 19 aa, artificial
  synthetic sequence
KKKKKKKKKKKKKKKKKKK

SEQ ID NO: 52 - protein, 20 aa, artificial
  synthetic sequence
KRKKRKKRKRSKRKKRKKRK

SEQ ID NO: 53 - protein, 21 aa, artificial
  synthetic sequence
KRKKRKKRKRSKRKKRKKRKK

SEQ ID NO: 54 - protein, 21 aa, artificial
  synthetic sequence
KRKKRKKRKKRKKRKKRKKRK

SEQ ID NO: 55 - protein, 22 aa, artificial
  synthetic sequence
KRKKRKKRKRGSGKRKKRKKRK

SEQ ID NO: 56 - protein, 24 aa, artificial
  synthetic sequence
KRKKRKKRKRGSGSGKRKKRKKRK

SEQ ID NO: 57 - protein, 25 aa, artificial
  synthetic sequence
KRKKRKKRKKRKKRKKRKKRKKRKK

SEQ ID NO: 58 - protein, 31 aa, artificial
  synthetic sequence
KRKKRKKRKRSKRKKRKKRKRSKRKKRKKRK

SEQ ID NO: 59 - protein, 38 aa, artificial
  synthetic sequence
KRKKRKKRKRGSGSGKRKKRKKRKGSGSGKRKKRKKRK

SEQ ID NO: 60 - protein, 39 aa, artificial
  synthetic sequence
KRKKRKKRKKRKKRKKRKKRKKRKKRKKRKKRKKRKKRK

SEQ ID NO: 61 - protein, 42 aa, artificial
  synthetic sequence
KRKKRKKRKRSKRKKRKKRKRSKRKKRKKRKRSKRKKRKKRK

SEQ ID NO: 62 - protein, 37 aa, Homo sapiens
LLGDFFRKSKEKIGKEFKRIVQRIKDFLRNLVPRTES

SEQ ID NO: 63 - protein, 29 aa, unknown
  SMAP-29 sheep
RGLRRLGRKIAHGVKKYGPTVLRIIRIAG

SEQ ID NO: 64 - protein, 13 aa, unknown
  Indolicidine bovine
ILPWKWPWWPWRR

SEQ ID NO: 65 - protein, 18 aa, unknown
  Protegrin Porcine
RGGRLCYCRRRFCVCVGR

SEQ ID NO: 66 - protein, 31 aa, unknown
  Cecropin P1 Mammal (pig)
SWLSKTAKKLENSAKKRISEGIAIAIQGGPR

SEQ ID NO: 67 - protein, 23 aa, unknown
  Magainin frog
GIGKFLHSAKKFGKAFVGEIMNS

SEQ ID NO: 68 - protein, 25 aa, unknown
  Pleurocidin fish
GWGSFFKKAAHVGKHVGKAALTHYL

SEQ ID NO: 69 - protein, 36 aa, Aedes aegypti
GGLKKLGKKLEGAGKRVFNAAEKALPVVAGAKALRK

SEQ ID NO: 70 - protein, 40 aa, Drosophila melanogaster
GWLKKIGKKIERVGQHTRDATIQGLGIPQQAANVAATARG

SEQ ID NO: 71 - protein, 21 aa, unknown
  Buforin II vertebrate
TRSSRAGLQFPVGRVHRLLRK

SEQ ID NO: 72 - protein, 39 aa, unknown
  Sarcotoxin IA Fly
GWLKKIGKKIERVGQHTRDATIQGLGIAQQAANVAATAR

SEQ ID NO: 73 - protein, 17 aa, Apis mellifera
ANRPVYIPPPRPPHPRL

SEQ ID NO: 74 - protein, 24 aa, unknown
  Ascaphine 5 Frog
GIKDWIKGAAKKLIKTVASHIANQ

SEQ ID NO: 75 - protein, 22 aa, unknown
  Nigrocine 2 Frog
GLLSKVLGVGKKVLCGVSGLVC

SEQ ID NO: 76 - protein, 24 aa, unknown
  Pseudin 1 Rana Frog
GLNTLKKVFQGLHEAIKLINNHVQ

SEQ ID NO: 77 - protein, 18 aa, unknown
  Ranalexin Frog
FLGGLIVPAMICAVTKKC

SEQ ID NO: 78 - protein, 26 aa, unknown
  Melittin bee
GIGAVLKVLTTGLPALISWIKRKRQQ

SEQ ID NO: 79 - protein, 25 aa, unknown
  Lycotoxin 1 Spider
IWLTALKFLGKHAAKKLAKQQLSKL

SEQ ID NO: 80 - protein, 19 aa, unknown
  Parasin 1 Fish
KGRGKQGGKVRAKAKTRSS

SEQ ID NO: 81 - protein, 39 aa, unknown
  Buforin I Toad
AGRGKQGGKVRAKAKTRSSRAGLQFPVGRVHRLLRKGNY

SEQ ID NO: 82 - protein, 34 aa, unknown
  Dermaseptin 1 Frog
ALWKTMLKKLGTMALHAGKAALGAAADTISQGTQ

SEQ ID NO: 83 - protein, 12 aa, unknown
  Bactenecin 1 Cow
RLCRIVVIRVCR

SEQ ID NO: 84 - protein, 21 aa, unknown
  Thanatin Insect
GSKKPVPIIYCNRRTGKCQRM

SEQ ID NO: 85 - protein, 19 aa, unknown
  Brevinin 1T Rana frogs
VNPIILGVLPKVCLITKKC

SEQ ID NO: 86 - protein, 26 aa, unknown
  Ranateurin 1 Rana frog
SMLSVLKNLGKVGLGFVACKINIKQC

SEQ ID NO: 87 - protein, 46 aa, unknown
  Esculentin 1 Rana frogs
GIFSKLGRKKIKNLLISGLKNVGKEVGMDVVRTGIKIAGCKIKGEC

SEQ ID NO: 88 - protein, 17 aa, Limulus polyphemus
RWCFRVCYRGICYRKCR

SEQ ID NO: 89 - protein, 25 aa, unknown
  Androctonin Scorpion
RSVCRQIKICRRRGGCYYKCTNRPY

SEQ ID NO: 90 - protein, 30 aa, Homo sapiens
DCYCRIPACIAGERRYGTCIYQGRLWAFCC

SEQ ID NO: 91 - protein, 38 aa, unknown
  beta-defensin cow
NPVSCVRNKGICVPIRCPGSMKQIGTCVGRAVKCCRKK

SEQ ID NO: 92 - protein, 18 aa, unknown
  theta-defensin monkey
GFCRCLCRRGVCRCICTR

SEQ ID NO: 93 - protein, 40 aa, unknown
  defensin (sapecin A) insect
ATCDLLSGTGINHSACAAHCLLRGNRGGYCNGKAVCVCRN

SEQ ID NO: 94 - protein, 46 aa, unknown
  Thionin (crambin) plant
TTCCPSIVARSNFNVCRIPGTPEAICATYTGCIIIPGATCPGDYAN

SEQ ID NO: 95 - protein, 50 aa, unknown
  defensin from radish
QKLCQRPSGTWSGVCGNNNACKNQCIRLEKARHGSCNYVFPAHCICYFPC

SEQ ID NO: 96 - protein, 30 aa, Bungarus fasciatus
KFFRKLKKSVKKRAKEFFKKPRVIGVSIPF

SEQ ID NO: 97 - protein, 44 aa, Drosophila melanogaster
DCLSGRYKGPCAVWDNETCRRVCKEEGRSSGHCSPSLKCWCEGC

SEQ ID NO: 98 - protein, 25 aa, Homo sapiens
DTHFPICIFCCGCCHRSKCGMCCKT

SEQ ID NO: 99 - protein, 44 aa, unknown
  Bac 5 Cow
RFRPPIRRPPIRPPFYPPFRPPIRPPIFPPIRPPFRPPLGRPFP

SEQ ID NO: 100 - protein, 39 aa, unknown
  PR-39 Pig
RRRPRPPYLPRPRPPPFFPPRLPPRIPPGFPPRFPPRFP

SEQ ID NO: 101 - protein, 20 aa, unknown
  Pyrrhocoricin Insect
VDKGSYLPRPTPPRPIYNRN

SEQ ID NO: 102 - protein, 24 aa, Homo sapiens
DSHAKRHHGYKRKFHEKHHSHRGY

SEQ ID NO: 103 - protein, 19 aa, Unknown
  ECP19
RPPQFTRAQWFAIQHISLN

SEQ ID NO: 104 - protein, 23 aa, Unknown
  MSI-594
GIGKFLKKAKKGIGAVLKVLTTG

SEQ ID NO: 105 - protein, 35 aa, Unknown
  TL-ColM
METLTVHAPSPSTNLPSYGNGAFSLSAPHVPGAGP

SEQ ID NO: 106 - protein, 18 aa, Unknown
  SBO
KLKKIAQKIKNFFAKLVA

SEQ ID NO: 107 - protein, 30 aa, Artificial sequence
  synthetic sequence
KFFRKLKKSVKKRAKRFFKKPRVIGVSIPF

SEQ ID NO: 108 - protein, 34 aa, Limulus polyphemus
GFKLKGMARISCLPNGQWSNFPPKCIRECAMVSS

SEQ ID NO: 109 - protein, 27 aa, artificial
  synthetic sequence
KRWVKRVKRVKRWVKRVVRVVKRWVKR

SEQ ID NO: 110 - protein, 25 aa, Artificial Sequence
  synthetic sequence; MW2
GKPGWLIKVALKFKKLIRRPLKRLA

SEQ ID NO: 111 - protein, 5 aa, Artificial sequence
  sequence, which is not part of the sequence motif, if the sequence motif contains at least three non-adjacent histidine residues
AALTH

SEQ ID NO: 112 - protein, 4 aa, Artificial sequence
  Example for intrasequential pairwise block of amino acids of the first group
LKRE

SEQ ID NO: 113 - protein, 5 aa, Artificial sequence
  Example for intrasequential triplet block of amino acids of the first group
LKRKE

SEQ ID NO: 114 - protein, 4 aa, Artificial sequence
  Example for N-terminal triplet block of amino acids of the first group
KRKE

SEQ ID NO: 115 - protein, 4 aa, Artificial sequence
  Example for C-terminal triplet block of amino acids of the first group
LKRK

SEQ ID NO: 116 - protein, 7 aa, Artificial sequence
  Example of a sequence precluded from the sequence motif, if a triplet of amnio acids of the first group is present
RRRGLRH

SEQ ID NO: 117 - protein, 4 aa, Artificial sequence
  Example of sequence not allowable within the sequence motif
KRKK

SEQ ID NO: 118 - protein, 4 aa, Artificial sequence
  Example of sequence not allowable within the sequence motif
RRRR

SEQ ID NO: 119 - protein, 18 aa, Unknown
  SMAP-29 sheep;aa1-18
RGLRRLGRKIAHGVKKYG

SEQ ID NO: 120 - protein, 25 aa, Artificial sequence
  Mutated peptide deriving from Cecropin A (A. aegypti)
GGLKKLGKKLKKAGKRVFKAAKKAL

SEQ ID NO: 121 - protein, 28 aa, Artificial sequence
  Mutated peptide deriving from BMAP-28
RGLRRLGRKILRAWKKYGPIIVPIIRIG

SEQ ID NO: 122 - protein, 17 aa, Artificial Sequence
  Mutated peptide deriving from MSI-78 (4-20)  peptide
RFLRRARRFGRAFVRIL

SEQ ID NO: 123 - protein, 26 aa, Artificial sequence
  Mutated peptide deriving from magainin
GIKKFLKSAKKFGKAFKKVIRGGGGS

SEQ ID NO: 124 - protein, 20 aa, Artificial sequence
  Mutated peptide deriving from HPA-NT3 peptide
KRLKKLAKKIWKWGRRGPGS

SEQ ID NO: 125 - protein, 29 aa, Artificial sequence
  Mutated peptide deriving from amino acids 298-326 of the alpha subunit of stonustoxin
IKLIKRVIKKFKKIFRKYPLTVKKGIAVG

SEQ ID NO: 126 - protein, 27 aa, Artificial sequence
  Mutated peptide deriving from amino acids 26-48 of CagL protein
GLKKLKRVYRKWVKAVKKVLKLGGGGS

SEQ ID NO: 127 - protein, 22 aa, Artificial sequence
  Mutated peptide deriving from amino acids 26-48 of CagL protein
GLKVLKKAYRRIRKAVRKILKA

SEQ ID NO: 128 - protein, 19 aa, Artificial sequence
  Mutated peptide deriving from amino acids 178-196 of IE1 protein
YKRAFKKVLKRIRRYAKRS

SEQ ID NO: 129 - protein, 24 aa, Artificial sequence
  synthetic sequence
GFFKKAWRKVKHAGRRVLKTAKGV

SEQ ID NO: 130 - protein, 20 aa, unknown
  CAP18AA
GLRKALRKFRNKIKEALKKI

SEQ ID NO: 131 - protein, 20 aa, artificial sequence
  synthetic sequence
GLRKALRKFRKKIKEALKKI

SEQ ID NO: 132 - protein, 29 aa, artificial sequence
  synthetic sequence
RGLRRLGRKIARGVKKYGPTVLRIIRIAG

SEQ ID NO: 133 - protein, 29 aa, Artificial sequence
  synthetic sequence
RGLRRLGRKIAHGVKKYGPTVLRHHHHHH

SEQ ID NO: 134 - protein, 29 aa, Artificial sequence
  synthetic sequence
RGIRKVLKFAKRLFRKIGRKPKGLIRVGA

SEQ ID NO: 135 - protein, 25 aa, Artificial sequence
  synthetic sequence
GRLFKRLAKKVAKTVRKFGRKIGAL

SEQ ID NO: 136 - protein, 29 aa, Pantholops hodgsoni
RGLRRLGRKILHGLKTYGPIVIPLIRLGG

SEQ ID NO: 137 - protein, 4 aa, artificial
  linker
GAGA

SEQ ID NO: 138 - protein, 8 aa, artificial
  linker
GAGAGAGA

SEQ ID NO: 139 - protein, 12 aa, artificial
  linker
GAGAGAGAGAGA

SEQ ID NO: 140 - protein, 195 aa, Artificial sequence
  Fusion of SMAP-29 peptide and the endolysin of Citrobacter koseri phage CkP1, with the additional technical modification of a C54S mutation
ARGLRRLGRKIAHGVKKYGPTVLRIIRIAGGSNIFKMLRIDEGYDSKIYKDTEGFWTIGIGHLLTRDPSLEVAKRELDKLVGRKSNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAYK

SEQ ID NO: 141 - protein, 195 aa, Artificial sequence
  Fusion of a peptide comprising the preferred sequence motif for the peptide component and the endolysin of Enterobacteria phage CC31, with the additional technical modification of a C54S mutation
ARGLRRLGRKIARGVKKYGPTVLRIIRIAGGSDIFGMLRIDEGYDSKIYKDTEGFWTIGIGHLLTRDPSLDVAKRELDKLVGRPSNGQITKAEAEAIFAKDVDKATRGILGNAVLKPVYDVLDGVRRAALINMVFQMGVAGVASFPASMRLLKSKQWEAAAKELANSKWYRQTPNRAKRVIATFKTGTWKAYENL

SEQ ID NO: 142 - protein, 195 aa, Artificial sequence
  Fusion of a peptide comprising the preferred sequence motif for the peptide component and the endolysin of Citrobacter koseri phage CkP1, with the additional technical modification of a C54S mutation
MRGLRRLGRKIAHGVKKYGPTVLRHHHHHHGSNIFKMLRIDEGYDSKIYKDTEGFWTIGIGHLLTRDPSLEVAKRELDKLVGRKSNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAYK

SEQ ID NO: 143 - protein, 195 aa, Artificial sequence
  Fusion of a peptide comprising the preferred sequence motif for the peptide component and the endolysin of Enterobacteria phage CC31, with the additional technical modification of a C54S mutation
ARGIRKVLKFAKRLFRKIGRKPKGLIRVGAGSDIFGMLRIDEGYDSKIYKDTEGFWTIGIGHLLTRDPSLDVAKRELDKLVGRPSNGQITKAEAEAIFAKDVDKATRGILGNAVLKPVYDVLDGVRRAALINMVFQMGVAGVASFPASMRLLKSKQWEAAAKELANSKWYRQTPNRAKRVIATFKTGTWKAYENL

SEQ ID NO: 144 - protein, 193 aa, Artificial sequence
  Fusion of a peptide according to SEQ ID NO 107  and the endolysin of Serratia phage CHI14, with the additional technical modification of a C54S mutation
AKFFRKLKKSVKKRAKRFFKKPRVIGVSIPFGSDIFGMLRIDEGYDSKIYKDTEGYWTIGIGHLLTKNPSLSVAKAELDKLVGRSSNGQITQDEAESIFAKDVEKAVKGIQGNSVLKPVYDSLDEIRRAALINMVFQMGVAGVAGFTNSMRMLKEKRWDEAAVNLAKSRWYNQTTNRAKRVISTFKTGTWGAY

SEQ ID NO: 145 - protein, 197 aa, Artificial sequence
  Fusion of a peptide comprising the preferred sequence motif for the peptide component and the endolysin of Aeromonas phage Ah1, with the additional technical modification of a C 122S mutation
ARGLRRLGRKIARGVKKYGPTVLRIIRIAGGSALAQMLKQDEGYKETVYWDTEGYPTIGIGHLILKKRTKDMGEINRELSSHVGRVVKDGKITGEEVLALFERDLSVLKRSIMSLPNLADVYVSLDMVRQTAIENMVFQMGAVGVSKFPGMLRSLKAKDWDGAYRNALDSAWARQTPNRAKRVASVLKLGSYAPYGF

SEQ ID NO: 146 - protein, 198 aa, Artificial sequence
  Fusion of a peptide according to SEQ ID NO 107  and the endolysin of Aeromonas phage Ah1, with the additional technical modification of a C122S mutation
AKFFRKLKKSVKKRAKRFFKKPRVIGVSIPFGSALAQMLKQDEGYKETVYWDTEGYPTIGIGHLILKKRTKDMGEINRELSSHVGRVVKDGKITGEEVLALFERDLSVLKRSIMSLPNLADVYVSLDMVRQTAIENMVFQMGAVGVSKFPGMLRSLKAKDWDGAYRNALDSAWARQTPNRAKRVASVLKLGSYAPYGF

SEQ ID NO: 147 - protein, 194 aa, Artificial sequence
  Fusion of a peptide comprising the preferred sequence motif for the peptide component and the endolysin of Serratia phage PS2
ARGLRRLGRKIARGVKKYGPTVLRIIRIAGGSATIFEMLAFDEGLKLTVYLDTEGFWTVGIGHLLTKNPSKAVAIAELDKLVGRSTGGTITKAEAERIFAQDVAKSEKGIQGNAVLGPVYAGLDATRKMALVNMTFQLGVAGAAGFTNSMKLLAAKQWKEAAINLAKSKWYNQTPNRAKRVISVFETGTLAAYK

SEQ ID NO: 148 - protein, 195 aa, Artificial sequence
  Fusion of peptide Cathelecidin-BF and the endolysin of Serratia phage PS2
AKFFRKLKKSVKKRAKEFFKKPRVIGVSIPFGSATIFEMLAFDEGLKLTVYLDTEGFWTVGIGHLLTKNPSKAVAIAELDKLVGRSTGGTITKAEAERIFAQDVAKSEKGIQGNAVLGPVYAGLDATRKMALVNMTFQLGVAGAAGFTNSMKLLAAKQWKEAAINLAKSKWYNQTPNRAKRVISVFETGTLAAYK

SEQ ID NO: 149 - protein, 190 aa, Artificial sequence
  Fusion of a peptide comprising the preferred sequence motif for the peptide component and the endolysin of Citrobacter koseri phage CkP1, with the additional technical modification of a C54S mutation
GRLFKRLAKKVAKTVRKFGRKIGALGSNIFKMLRIDEGYDSKIYKDTEGFWTIGIGHLLTRDPSLEVAKRELDKLVGRKSNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAYK

SEQ ID NO: 150 - protein, 195 aa, Artificial sequence
  Fusion of a peptide comprising the preferred sequence motif for the peptide component and the endolysin of Citrobacter koseri phage CkP1, with the additional technical modification of a C54S mutation
ARGLRRLGRKILHGLKTYGPIVIPLIRLGGGSNIFKMLRIDEGYDSKIYKDTEGFWTIGIGHLLTRDPSLEVAKRELDKLVGRKSNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAYK

SEQ ID NO: 151 - protein, 198 aa, Artificial sequence
  Fusion of a peptide comprising the preferred sequence motif for the peptide component and the endolysin of  Aeromonas phage As-szw
ARGLRRLGRKIARGVKKYGPTVLRIIRIAGGSALEKMLKFDEGSKLSVYWDTEGYPTIGIGHLIKRLRTKDMGEINRELSSHVGRVITDGKITQSEESQLFAKDLEVVRNSMKGYVDLWSTYVGLDEVRKTALENMVFQMGAKGVNGFPSMLRAMRSKNWVEAKKHGLASAWSRQTPNRAKRVTDVIETGTYKGYPFA

SEQ ID NO: 152 - protein, 192 aa, Artificial Sequence
  Fusion of a peptide comprising the preferred sequence motif for the peptide component and the endolysin of Serratia phage CHI14, with the additional technical modification of a C54S mutation
ARGLRRLGRKIARGVKKYGPTVLRIIRIAGGSDIFGMLRIDEGYDSKIYKDTEGYWTIGIGHLLTKNPSLSVAKAELDKLVGRSSNGQITQDEAESIFAKDVEKAVKGIQGNSVLKPVYDSLDEIRRAALINMVFQMGVAGVAGFTNSMRMLKEKRWDEAAVNLAKSRWYNQTTNRAKRVISTFKTGTWGAY

SEQ ID NO: 153 - protein, 6 aa, artificial sequence
  His-Tag (6x)
HHHHHH

SEQ ID NO: 154 - protein, 196 aa, Artificial sequence
  A fusion of Cecropin A. (A aegyptii) peptide with endolysin of Vibrio phage VvAW1(YP_007518361.1)
MGGLKKLGKKLEGAGKRVFNAAEKALPVVAGAKALRKGGGSGGGSGSMGFKFSERSKSRMAGVHPELVLVFHEALAVSPIDFGIPEHGGLRSAEEQYSLFLDNKSKADGYNKLSNHQSGNALDFYAYLNGAASWDKVHLAMVAATILSTAARLKEQGKISISIRWGGTFGNKGRSFHGWDYPHMEVISLEHHHHHH

SEQ ID NO: 155 - protein, 295 aa, Artificial sequence
  A fusion of Cecropin A. (A aegyptii) peptide with a mutated cell wall binding domain of the modular KZ144 endolysin and Lys68 endolysin
MGGLKKLGKKLEGAGKRVFNAAEKALPVVAGAKALRKGAGAGAGAGSKVLRKGDRGDEVSQLQTLLNLSGYDVGKPDGIFGNNTFNQVVKFQKDNSLDSDGIVGKNTWAELFSKYSPPIPYKTIPMSNRNISDNGIKFTAAFEGFRGTAYRATKNEKYLTIGYGHYGADVKEGQKITEGQGLLLLHKDMVKAVAAVDAVAHPPLNQSQFDAMCDLVYNAGVGVIAASTGTGQALRKGDVATLRNKLTQFHYQNGKSLLGLRRRAAGRVALFDGMLWQQAEAIGRGAKLEHHHHHH

SEQ ID NO: 156 - protein, 223 aa, Artificial sequence
  Fusion of a modified peptide (SEQ ID NO110) and endolysin of Pseudomonas phage vB_PsyM_KIL1
MGLRKALRKFRKKIKEALKKIGGGGSGSMLSEKSFVEAAASLGCEVAAIKAIASVETKGSAWITPGVPQILYERHIMARLLKAKGVPIAGLPSDLVNTTPGGYGKFSEQHGKLDRAVKIDRECALQSCSWGMFQLMGFNYKLCGYATVQAFVNAMYKSEDEQLNAFVGFIKSNLQLNDALKSKDWATVARLYNGADYKINSYDQKLAVAYESNKRLEHHHHHH